(19) United States
(12) Patent Application Publication
(10) Pub. No.: US 2021/0038648 A1
(43) Pub. Date: Feb. 11, 2021

(54) ANTI-GUCY2C CHIMERIC ANTIGEN RECEPTOR COMPOSITIONS AND METHODS

(71) Applicant: THOMAS JEFFERSON UNIVERSITY, Philadelphia, PA (US)

(72) Inventors: Scott A. Waldman, Philadelphia, PA (US); Adam Snook, Aston, PA (US); Trevor R Baybutt, Dresher, PA (US); Michael Magee, Cambridge, MA (US)

(21) Appl. No.: 16/981,278

(22) PCT Filed: Mar. 16, 2019
(86) PCT No.: PCT/US2019/022645

§ 371 Date: Sep. 15, 2020

Related U.S. Application Data
(60) Provisional application No. 62/643,850, filed on Mar. 16, 2018.

(51) Int. Cl. A61K 35/17; C07K 19/00; C07K 14/535; A61K 38/20; A61P 35/00

(52) U.S. Cl. CPC A61K 35/17; C07K 19/00; C07K 14/535; C07K 2317/52; A61P 35/00; C07K 2317/569; A61K 38/2013

(57) ABSTRACT

Proteins comprising anti-GUCY2C scFvs and nucleic acid molecules that encode anti-GUCY2C scFvs are disclosed. Proteins comprising signal sequence linked to anti-GUCY2C scFvs linked to hinge, transmembrane and signal domain sequences are disclosed. Nucleic acid molecules that encode proteins comprising signal sequence linked to anti-GUCY2C scFvs linked to hinge, transmembrane and signal domain sequences are disclosed. T cells that comprise such proteins and such nucleic acid molecules that are disclosed. Methods of making the T cells and methods of using the T cells to treat or prevent cancer that has cancer cells that express GUCY2C are disclosed.

### FIELD OF THE INVENTION

The invention relates to chimeric antigen receptors that bind to guanylyl cyclase C and nucleic acid molecules that encode such chimeric antigen receptors. The invention also relates to cells that comprise such chimeric antigen receptors, to methods of making such chimeric antigen receptors and cells, and to methods of using such cells to treat individuals who are suffering from cancer that has cancer cells which express guanylyl cyclase C and to protect individuals against cancer that has cancer cells which express guanylyl cyclase C.

### BACKGROUND OF THE INVENTION

Immunotherapy based upon T cells that express chimeric antigen receptors (CARs) has become an emerging modality for treating cancer. CARs are fusion receptors that comprise a domain which functions to provide HLA-independent binding of cell surface target molecules and a signaling domain that can activate host immune cells of various types, typically peripheral blood T cells, which may include populations of cells referred to cytotoxic lymphocytes, cytotoxic T lymphocytes (CTLs), Natural Killer T cells (NKT) and Natural Killer cells (NK) or helper T cells. That is, while typically being introduced into T cells, genetic material encoding CARs may be added to immune cells that are not T cells such as NK cells.

Guanylyl cyclase C (also referred to interchangeably as GCC or GUCY2C) is a membrane-bound receptor that produces the second messenger cGMP following activation by its hormone ligands guanylin or uroguanylin, regulating intestinal homeostasis, tumorigenesis, and obesity. GUCY2C cell surface expression is confined to luminal surfaces of the intestinal epithelium and a subset of hypothalamic neurons. Its expression is maintained in >95% of colorectal cancer metastases and it is ectopically expressed in tumors that evolve from intestinal metaplasia, including esophageal, gastric, oral, salivary gland and pancreatic cancers.

The inaccessibility of GUCY2C in the apical membranes of polarized epithelial tissue due to subcellular restriction of GUCY2C, creates a therapeutic opportunity to target metastatic lesions of colorectal origin which have lost apical-basolateral polarization, without concomitant intestinal toxicity.

A syngeneic, immunocompetent mouse model demonstrated that CAR-T cells targeting murine GUCY2C were effective against colorectal cancer metastatic to lung in the absence of intestinal toxicities. Similarly, other GUCY2C-targeted therapeutics, including antibody-drug conjugates and vaccines, are safe in preclinical animal models, and therapeutic regimens utilizing these platforms are in clinical trials for metastatic esophageal, gastric, pancreatic, and colorectal cancers (NCT02202759, NCT02202785, NCT01972737).

The safety of these therapeutic regimens, in the context of GUCY2C expression across the rostral-caudal axis of intestine, reflects compartmentalized expression of GUCY2C, enriched in apical, but limited in basolateral, membranes of epithelial cells. Systemic radiolabeled imaging agents conjugated to GUCY2C ligand target GUCY2C-expressing metastases without localizing in intestine, confirming the mucosal compartmentalization of the receptor.

Tumors express up to 10-fold greater amounts of GUCY2C, compared to normal epithelial cells, potentially creating a quantitative therapeutic window to discriminate receptor overexpressing tumors from intestinal epithelium with low/absent GUCY2C in basolateral membranes.

U.S. Patent Application Publication 20120251509 A1 and U.S. Patent Application Publication US 2014-0294784 A1, which are each incorporated herein by reference, disclose CARs including CARs that bind to guanylyl cyclase C, T cells that comprise CARs including T cells that comprise CARs that bind to GUCY2C and target cells that comprise GUCY2C, methods of making chimeric antigen receptors and T cells, and methods of using T cells that comprise CARs that bind to GUCY2C and target cells that comprise GUCY2C to protect individuals against cancer cells that express GUCY2C and to treat individuals who are suffering from cancer in which cancer cells express GUCY2C.

There is remains a need for improved compositions and methods to protect individuals against cancer cells that express GUCY2C and to treat individuals who are suffering from cancer in which cancer cells express GUCY2C.

### SUMMARY OF THE INVENTION

Proteins comprising an anti-GUCY2C scFV sequence are provided. The anti-GUCY2C scFV sequences may be selected from the group consisting of SEQ ID NO:8, SEQ ID NO:9, SEQ ID NO:10, SEQ ID NO:11, SEQ ID NO:12, SEQ ID NO:13, SEQ ID NO:14 and SEQ ID NO:15.

Proteins comprising the 5F9 anti-GUCY2C scFV sequence and further comprising a signal sequence, a hinge domain, a transmembrane domain, and a signaling domain are provided.

Nucleic acid molecules that encode such proteins are provided. The nucleic acid molecules may be operably linked to regulatory elements that can function to express the protein in a human cell such as a human T cell. The nucleic acid molecules may be incorporated in a nucleic acid vector such as a plasmid or recombinant viral vector that can be used transform human cells into human cells that express the protein.

Human cells comprising the nucleic acid molecules and express the proteins are provided.

Methods of making such cells are provided.

Methods of treating a patient who has cancer that has cancer cells that express GUCY2C and methods of preventing cancer that has cancer cells that express GUCY2C in a patient identified as being of increased risk, are provided.

### BRIEF DESCRIPTION OF FIGURES

FIG. 1 panels A-E. Generation of human GUCY2C-specific CAR-T cells. FIG. 1 panel A: Recombinant 5F9 antibody was assessed by ELISA for specific binding to hGUCY2CECD or BSA (negative control) plated at 1 μg/mL. Two-way ANOVA; ****p<0.0001. FIG. 1 panel B: Flow cytometry analysis was performed on parental CT26 mouse colorectal cancer cells or CT26 cells engineered to express hGUCY2C (CT26.hGUCY2C) and stained with 5F9 antibody. FIG. 1 panel C: Schematic of the third generation murine CAR construct containing murine sequences of the BiP signal sequence, 5F9 scFv, CD8α hinge region, the transmembrane and intracellular domain of CD28, the intracellular domain of 4-1BB (CD137), and the intracellular domain of CD3ζ (5F9.m28BBz). The CAR construct was inserted into the MSCV retroviral plasmid pMIG upstream of an IRES-GFP marker. FIG. 1 panel D: Murine CD8+ T cells transduced with a retrovirus containing a control (1D3.m28BBz) CAR or CAR derived from the 5F9 antibody (5F9.m28BBz) were labeled with purified 6×His-hGUCY2CECD (10 μg/mL), detected with anti-5×His-Alexa Fluor 647 conjugate. Flow plots were gated on live CD8+ cells. FIG. 1 panel E: 6×His-hGUCY2CECD binding curves for 5F9-derived or control (1D3) CARs, gated on live CD8+GFF+ cells (See data in FIG. 5). Combined from 3 independent experiments.

FIG. 2 panels A-E. hGUCY2C-specific CARs mediate antigen-dependent T-cell activation and effector functions. In FIG. 2 panels A-E, Murine CD8+ T cells were left non-transduced (None) or transduced with control 1D3.m28BBz or 5F9.m28BBz CAR constructs as indicated. FIG. 2 panel A: Gating strategy for all analyses in FIG. 2 panels B-D. FIG. 2 panel B: Representative CAR-T cell phenotyping plot based on CD45RA and CD62L. Two-way ANOVA; NS: not significant; Bars: mean±SD from 2-3 independent experiments; Tn/scm; naïve or T memory stem cells; Tcm; central memory T cells; Tem: effector memory T cells; Temra: effector memory T cells expressing CD45RA. (C-D) 10⁶ CAR-T cells were stimulated for 6 hours with plate-coated antigen (BSA or hGUCY2C) or PMA and ionomycin (PMA/IONO). T-cell activation markers (CD25, CD69, or CD44) and intracellular cytokine production (IFNγ, TNFα, IL2, and MIP1α) were then quantified by flow cytometry. Graphs indicate the mean±SD. FIG. 2 panel C refers to activation marker upregulation (MFI) and FIG. 2 panel D refers to polyfunctional cytokine production (% of CAR+ cells) from 3 independent experiments. FIG. 2 panel E: Parental CT26 or CT26.hGUCY2C mouse colorectal cancer cells in an E-Plate were treated with CAR-T cells (5:1 E:T ratio), media, or 10% Triton-X 100 (Triton), and the relative electrical impedance was quantified every 15 minutes for 10 hours to quantify cancer cell death (normalized to time=0). Percent specific lysis values were calculated using impedance values following the addition of media and Triton for normalization (0% and 100% specific lysis, respectively). Two-way ANOVA, B-E; *p<0.05, **p<0.01, ***p<0.001, ****p−0.0001.

FIG. 3 panels A-E. hGUCY2C CAR-T cells provide long-term protection in a syngeneic lung metastasis model. In FIG. 3 panels A-E, BALB/c mice were injected with 5×10⁵ CT26.hGUCY2C cells via the tail vein to establish lung metastases. Control (4D5.m28BBz) or 5F9.m28BBz CAR constructs were transduced into murine CD8+ T cells. FIG. 3 panel A: Mice were treated 3 days later with 5 Gy total body irradiation (TBI) followed by 10⁶-10⁷ 5F9.m28BBz (N=7-8/group) or 10⁷ control (N=6) CART cells. FIG. 3 panel B: Mice were treated on day 3 (D3) or day 7 (D7) with 5 Gy TBI followed by 10⁷ control (N=10/group) or 5F9.m28BBz (N=9-10/group) CAR-T cells. FIG. 3 panel C: Mice were treated on day 7 with 5 Gy TBI followed by 10⁷ control (N=10) or 5F9.m28BBz (N=12) CAR-T cells on day 7 and day 14. FIG. 3 panel D: Mice treated on day 7 with 5 Gy TBI and PBS or 10⁷ control or 5F9.m28BBz CAR-T cells were sacrificed on day 18. lungs sunned with India ink, and tumors/lung enumerated. One-way ANOVA; *p<0.05. FIG. 3 panel E: Surviving mice from B and C treated with 5F9.m28BBz CAR-T cells or naïve mice were challenged with 5×10⁵ CT26 (N=4-7/group) or CT26.hGUCY2C (N=7/group) cells (re-challenge occurred 16-40 weeks after initial challenge). Log-rank Mantel-Cox test. FIG. 3 panels A-C and E; **p<0.01, ***p<0.001, ****p<0.0001. Up arrows indicate CAR-T cell treatment days. Each panel indicates an independent experiment

FIG. 4 panels A-E. hGUCY2C CAR-T cells eliminate human colorectal tumor xenografts. FIG. 4 panel A: hGUCY2C expression on T84 human colorectal cancer cells was quantified by flow cytometry using the recombinant 5F9 antibody. In FIG. 4 panels B-E, Control (1D3.m28BBz) or 5F9.m28BBz CAR constructs were transduced into murine CD8+ T cells. FIG. 4 panel B: T84 colorectal cancer cells in an E-Plate were treated in duplicate with 5F9-m28BBz or control CAR-T cells (5:1 E:T ratio), media, or 10% Triton-X 100 (Triton), and the relative electrical impedance was measured every 15 minutes for 20 hours to quantify cancer cell death (normalized to time=0). Percent specific lysis values were calculated using impedance values following the addition of media and Triton for normalization (0% and 100% specific lysis, respectively). Two-way ANOVA; **p<0.01; representative of two independent experiments. In FIG. 4 panels C-E. Immunodeficient NSG mice were injected with 2.5×10⁶ luciferase-expressing T84 colorectal cancer cells via intraperitoneal injection and were treated with 10⁷ control (N=5) or 5F9-m28BBz (N=4) CAR-T cells on day 14 by intraperitoneal injection. In FIG. 4 panels C-D, Total tumor luminescence (photons/second) was quantified just prior to T-cell injection and weekly thereafter. Two-way ANOVA; *p<0.05. FIG. 4 panel E: Mice were followed for survival. Log-rank Mantel Cox test; *p<0.05.

FIG. 5. Detection of 5F9.m28BBz CAR surface expression. Murine CD8+ T cells transduced with a retrovirus containing a control m28BBz CAR or CAR derived from the 5F9 antibody (5F9.m28BBz) upstream of an IRES-GFP marker were labeled with purified 6×HishGUCY2CECD (0-1430 nM) and detected with α5×His-Alexa-647 conjugate. Flow plots were gated on live CD8+ cells.

FIG. 6. hGUCY2C-expressing mouse colorectal cancer cells activate 5F9.m28BBz CAR-T cells. 10⁶ CAR-T cells were stimulated for 6 h with 10⁶ parental CT26, CT26.hGUCY2C colorectal cancer cells or PMA and ionomycin (PMA/IONO). T-cell activation markers (CD25, CD69, or CD44) were quantified by flow cytometry.

FIG. 7, panels A and B. hGUCY2C-expressing mouse colorectal cancer cells induce 5F9.m28BBz CAR-T cell cytokine production. 10⁶ CAR-T cells were stimulated for 6 h with plate-coated antigen. FIG. 7, panel A shows data for BSA, hGUCY2C, and PMA and ionomycin (PMA/IONO). FIG. 7, panel B shows data for 10⁶ parental CT26 or CT26.hGUCY2C colorectal cancer cells or PMA and ionomycin (PMA/IONO). Intracellular cytokine production (IFNγ, TNFα, IL-2 or MIP1α) was quantified by flow cytometry.

FIG. 8 panels A and B. 5F9.m28BBz CAR-T cells kill hGUCY2C-expressing mouse colorectal cancer cells. β-galactosidase-expressing CT26 (data in FIG. 8 panel A) or CT26.hGUCY2C (data in FIG. 8 panel B) mouse colorectal cancer cells were cultured for 4 h with a range of effector CAR-T cell:target cancer cell ratios (E:T Ratio). Specific lysis was determined by β-galactosidase release into the supernatant detected by a luminescent substrate. ****, p<0.0001 (Two-way ANOVA).

FIG. 9 panels A and B. 5F9.m28BBz CAR-T cells do not kill hGUCY2C-deficient human colorectal tumors. FIG. 9, panel A: hGUCY2C expression on SW480 human colorectal cancer cells was quantified by flow cytometry using the recombinant 5F9 antibody. FIG. 9, panel B: SW480 cells in an E-Plate were treated with 5F9.m28BBz or control 1D3.m28BBz CAR T cells, media, or 2.5% Triton-X 100 (Triton) and the relative electrical impedance was quantified every 15 min for 20 h to quantify cancer cell death (normalized to time=0). Percent specific lysis values were calculated using impedance values following the addition of media and Triton for normalization (0% and 100% specific lysis, respectively).

FIG. 10 panels A-C. Human T cells expressing 5F9.h28BBz CAR recognize and kill GUCY2C-expressing colorectal cancer cells. FIG. 10 panel A: CAR-T cells expressing a human 5F9 CAR construct (5F9.h28BBz) were stimulated for 6 hours with plate-coated antigen (BSA or hGUCY2C) or PMA and ionomycin (PMA/IONO). The T-cell activation marker CD69 and intracellular cytokines (IFNγ, TNFα, and IL-2)□ were then quantified by flow cytometry. In reference to data in FIG. 10 panels B-C, Parental (CT26), human GUCY2Cexpressing CT26 (CT26.hGUCY2C) mouse colorectal cancer cells (data shown in FIG. 10 panel B), or T84 human colorectal cancer cells (data shown in FIG. 10 panel C) cultured in an E-Plate were treated with Control or 5F9.h28BBz CAR-T cells (E:T ratio of 10:1), media, or 2.5% Triton-X 100 and the relative electrical impedance was quantified every 15 min to quantify cancer cell death (normalized to time=0). Percent specific lysis values were calculated using impedance values following the addition of media and Triton for normalization (0% and 100% specific lysis, respectively). ***, p<0.001 (Two-way ANOVA).

FIG. 11 panels A and B. 5F9.m28BBz CAR-T cells do not kill mGUCY2C-expressing mouse colorectal cancer cells. CT26 cells expressing β-galactosidase and murine GUCY2C (FIG. 11 panel A; CT26.mGUCY2C) or human GUCY2C (FIG. 11 panel B; CT26.hGUCY2C) were cultured for 4 h with a range of effector CAR-T cell:target cancer cell ratios (E:T Ratio). Specific lysis was determined by β-galactosidase release into the supernatant detected by a luminescent substrate. ****, p<0.0001 (Two-way ANOVA).

### DETAILED DESCRIPTION OF THE PREFERRED EMBODIMENTS

Single chain protein sequences that bind to the extracellular domain of human GUCY2C were generated using fragments of the variable light chain and variable heavy chain of an anti-GUCY2C antibody that binds to the extracellular domain of human GUCY2C. A linker sequence connects the variable light chain fragment to the variable heavy chain fragment into a single chain antibody variable fragment fusion protein sequence (scFv) that binds to the extracellular domain of human GUCY2C.

The scFv is a component in a CAR, which is a larger fusion protein. The CARs functional components include the immunoglobulin-derived antigen binding domain, antibody sequences i.e. svFv, which binds to human GUCY2C, a hinge domain that links the scFV to a transmembrane domain that anchors the protein in the cell membrane of the cell in which it is expressed, and the signally domain which functions as signal transducing intracellular sequences (also referred to as cytoplasmic sequences) that activate the cell upon scFv binding to human GUCY2C. The nucleic acid sequences that encode the CAR include sequences that encode a signal peptide from a cellular protein that facilitate the transport of the translated CAR to the cell membrane. CARs direct the recombinant cells in which they are expressed to bind to and, in the case of recombinant cytotoxic lymphocytes, recombinant cytotoxic T lymphocytes (CTLs), recombinant Natural Killer T cells (NKT), and recombinant Natural Killer cells (NK) kill cells displaying the antibody-specified target, i.e. GUCY2C. When the CAR is expressed it is transported to the cell surface and the signal peptide is typically removed. The mature CAR functions as a cellular receptor. The scFv and hinge domain are displayed on the cell surface where the scFv sequences can be exposed to proteins on other cells and bind to GUCY2C on such cells. The transmembrance region anchors the CAR in the cell membrane and the intracellular sequences function as a signal domain to transduce a signal in the cell which results in the death of GUCY2C-expressing cell to which the CAR-expressing cell is bound.

In some embodiments, the CARs comprise a signal sequence, such as for example a mammalian or synthetic signal sequence. In some embodiments, the CARs comprise a signal sequence from a membrane-bound protein such as for example a mammalian membrane-bound protein. In some embodiments, the CARs comprise a signal sequence from a membrane-bound protein such as CD8 alpha, CD8 beta, CD4, TCR alpha, TCR beta, CD3 delta, CD3 epsilon, CD3 gamma, CD28, and BiP. Examples of signal sequences may also be found in membrane bound any mammalian signal sequence <http://www.signalpeptide.de/index.php?m=listspdb_mammalia>. In some embodiments, the CARs comprise a Granulocyte-Macrophage Colony-Stimulating Factor (GM-CSF) signal sequence. In some embodiments, the CARs comprise a Granulocyte-Macrophage Colony-Stimulating Factor (GM-CSF) signal sequence having amino acids 1-22 of SEQ ID NO:2. In some embodiments, the Granulocyte-Macrophage Colony-Stimulating Factor (GM-CSF) signal sequence comprises amino acids 1-22 of SEQ ID NO:2. In some embodiments, the Granulocyte-Macrophage Colony-Stimulating Factor (GM-CSF) signal sequence consists essentially of amino acids 1-22 of SEQ ID NO:2. In some embodiments, the Granulocyte-Macrophage Colony-Stimulating Factor (GM-CSF) signal sequence consists of amino acids 1-22 of SEQ ID NO:2. In some embodiments, the nucleic acid sequence of the construct that encodes the CARs that comprise a Granulocyte-Macrophage CoIony-Stimulating Factor (GM-CSF) signal sequence comprise nucleic acid 1-66 of SEQ ID NO:1. In some embodiments, the nucleic acid sequence that encodes the Granulocyte Macrophage Colony-Stimulating Factor (GM-CSF) signal sequence comprises nucleic acid 1-66 of SEQ ID NO:1. In some embodiments, the nucleic acid sequence that encodes the Granulocyte-Macrophage Colony-Stimulating Factor (GM-CSF) signal sequence consists essentially of nucleic acid 1-66 of SEQ ID NO:1. In some embodiments, the nucleic acid sequence that encodes the Granulocyte-Macrophage Colony-Stimulating Factor (GM-CSF) signal sequence consists of nucleic acid 1-66 of SEQ ID NO:1.

The anti-GUCY2C binding domain is provided as a single chain chimeric receptor that is MHC-independent. The antigen-binding domain is derived from an antibody. In some embodiments, CARs comprise anti-guanylyl cyclase C (also referred to as GCC or GUCY2C) single chain variable fragment (scFv) (preferably a Variable Light fragment—(Glycine₄Serine)₄ Linker—Variable Heavy fragment) from 5F9. 5F9 is a hybridoma expressing a fully humanized, monoclonal antibody that recognizes the extracellular domain of human GUCY2C. The DNA coding sequences of the antibody heavy and light chains were used to create a novel scFv for CAR implementation that is employed in the creation of anti-GCC CARS, such as for example the 5F9-28BBz CAR, and confers antigen specificity directed towards the GUCY2C molecule.

In some embodiments such as the 5F9-28BBz CAR, the anti-GCC scFv may be a 5F9 single chain variable fragment (scFv) (Variable Light fragment—(Glycine₄Serine)₄ Linker—Variable Heavy fragment). The 5F9 say may comprise amino acids 25-274 of SEQ ID NO:2. In some embodiments, the nucleic acid sequence of the construct that encodes the CARs that comprise the 5F9 scFv comprise nucleotides 73-822 of SEQ ID NO:1. In some embodiments, the CARs comprise an anti-GCC 5-F9 scFv. Amino acids 25-133 of SEQ ID NO:2 corresponds to the 5F9 Variable Light chain fragment. Amino acids 154-274 of SEQ ID NO:2 corresponds to the 5F9 Variable Heavy chain fragment. In some embodiments, the CARs comprise an anti-GCC 5F9 single chain variable fragment (scFv) that corresponds to the 5F9 Variable Light fragment and the 5F9 Variable Heavy fragment attached to each other with a (Glycine₄Serine)ₙ LINKER in which (Glycine₄Serine)=GGGGS (SEQ ID NO:3) and n=2-5.

In some embodiments, the linker contains two (Glycine₄Serine) units ((Glycine₄Serine)₂) and may referred to as LINKER G4S-2 (SEQ ID NO:4). In some embodiments, the linker contains three (Glycine₄Serine) units ((Glycine₄Serine)₃) and may referred to as LINKER G4S-3 (SEQ ID NO:5). In some embodiments, the linker contains four (Glycine₄Serine) units ((Glycine₄Serine)₄) and may referred to as LINKER G4S-4 (SEQ ID NO:6). In some embodiments, the linker contains five (Glycine₄Serine) units ((Glycine₄Serine)₅) and may referred to as LINKER G4S-5 (SEQ ID NO:7).

The 5F9 variable fragments may be configured from N-terminus to C-terminus in the order Variable Light Chain fragment-LINKER-Variable Heavy Chain fragment or Variable Heavy Chain fragment-LINKER-Variable Light Chain fragment. In some embodiments, the CARs comprise an anti-GCC 5F9 scFv configured as [5F9 Variable Light Chain fragment—(Glycine₄Serine)₂-5F9 Variable Heavy Chain fragment] (SEQ ID NO:8), [5F9 Variable Light Chain fragment—(Glycine₄Serine)₃-5F9 Variable Heavy Chain fragment] (SEQ ID NO:9), [5F9 Variable Light Chain fragment—(Glycine₄Serine)₄-5F9 Variable Heavy Chain fragment] (SEQ ID NO:10), or [5F9 Variable Light Chain fragment—(Glycine₄Serine)₅-5F9 Variable Heavy Chain fragment] (SEQ ID NO:11). In some embodiments, the CARs comprise an anti-GCC 5F9 scFv configured as [5F9 Variable Heavy Chain fragment—(Glycine₄Serine)₂-5F9 Variable Light Chain fragment] (SEQ ID NO:12), [5F9 Variable Heavy Chain fragment—(Glycine₄Serine)₃-5F9 Variable Light Chain fragment] (SEQ ID NO:13), [5F9 Variable Heavy Chain fragment—(Glycine₄Serine)₄-5F9 Variable Light Chain fragment] (SEQ ID NO:14), or [5F9 Variable Heavy Chain fragment—(Glycine₄Serine)₅-5F9 Variable Light Chain fragment (SEQ ID NO:15).

In some embodiments, the CARs comprise an anti-GCC 5F9 scFv having amino acids 25-274 of SEQ ID NO:2. In some embodiments, the 5F9 scFv comprises amino acids 25-274 SEQ ID NO:2. In some embodiments, the 5F9 scFv consists essentially of amino acids 25-274 of SEQ ID NO:2. In some embodiments, the 5F9 scFv consists of amino acids 25-274 of SEQ ID NO:2. In some embodiments, the nucleic acid sequence that encodes the 5F9 scFv comprises nucleotides 73-822 of SEQ ID NO:1. In some embodiments, the nucleic acid sequence that encodes the 5F9 scFv consists essentially of nucleotides 73-822 of SEQ ID NO:1. In some embodiments, the nucleic acid sequence that encodes the 5F9 scFv consists of nucleotides 73-822 of SEQ ID NO:1.

In some embodiments, CARs comprise a CD8α, IgG1-Fc, IgG4-Fc, or CD28 hinge region. In some embodiments, CARs comprise a CD8α hinge region. In some embodiments, CARs comprise a CD8α hinge region having amino acids 277-336 of SEQ ID NO:2. In some embodiments, the CD8α hinge region comprises amino acids 277-336 of SEQ ID NO:2. In some embodiments, the CD8α hinge region consists essentially of amino acids 27-336 of SEQ ID NO:2. In some embodiments, the CD8α hinge region consists of amino acids 277-336 of SEQ ID NO:2. In some embodiments, the nucleic acid sequence that encodes the CD8α hinge region comprises nucleotides 829-1008 of SEQ ID NO:1. In some embodiments, the nucleic acid sequence that encodes the CD8α hinge region consists essentially of nucleotides 829-1008 of SEQ ID NO:1. In some embodiments, the nucleic acid sequence that encodes the CD8α hinge region consists of nucleotides 829-1008 of SEQ ID NO:1.

In some embodiments, CAR s comprise a CD28, 4-1BB (CD137), CD2, CD27, CD30, CD40L, CD79A, CD79B, CD226, DR3, GITR, HVEM, ICOS, LIGHT, OX40, or SLAM transmembrane region.

In some embodiments, CARs comprise a CD28, 4-1BB (CD137) CD2, CD27, CD30, CD40L, CD79A, CD79B, CD226, DR3, GITR, HVEM, ICOS, OX40, or SLAM intracellular region.

In some embodiments, CARs comprise both transmembrane and intracellular (cytoplasmic) sequences from CD28, 4-1BB (CD137), CD2, CD27, CD30, CD40L, CD79A, CD79B, CD226, DR3, GITR, HVEM, ICOS, LIGHT, OX40, or SLAM. In some embodiments, CARs comprise CD28 transmembrane and intracellular sequences. In some embodiments, CARs comprise CD28 transmembrane and intracellular sequences having amino acids 337-405 of SEQ ID NO:2. In some embodiments, the CD28 transmembrane and intracellular sequences comprises amino acids 337-405 of SEQ ID NO:2. In some embodiments, the CD28 transmembrane and intracellular sequences consists essentially of amino adds 337-405 of SEQ ID NO:2. In some embodiments, the CD28 transmembrane and intracellular sequences consists of amino acids 337-405 of SEQ ID NO:2. In some embodiments, the nucleic acid sequence that encodes CD28 transmembrane and intracellular sequences comprises nucleotides 1009-1215 of SEQ ID NO:1. In some embodiments, the nucleic acid sequence that encodes CD28 transmembrane and intracellular sequences consists essentially of nucleotides 1009-1215 of SEQ ID NO:1. In some embodiments, the nucleic acid sequence encodes CS28 transmembrane and intracellular sequences consists of nucleotides 1009-1215 of SEQ ID NO:1.

In some embodiments, CARs comprise intracellular (cytoplasmic) sequences from ζ-chain associated with CD3 (CD3ζ), the CD79-alpha and -beta chains of the B cell receptor complex, or certain Fc receptors.

In some embodiments, CARs comprise a) intracellular (cytoplasmic) sequences from one or more of CD28, 4-1BB (CD137), CD2, CD27, CD30, CD40L, CD79A, CD79B, CD226, DR3, GITR, HVEM, ICOS, LIGHT, OX40. or SLAM intracellular region in combination with b) intracellular (cytoplasmic) sequences from ζ-chain associated with CD3 (CD3ζ), the CD79-alpha and -beta chains of the B cell receptor complex, or certain Fc receptors.

In some embodiments, CARs comprise CD28 transmembrane and intracellular sequences together with 4-1BB intracellular sequences in combination with CD3ζ intracellular sequences.

In some embodiments, CARs comprise CD28 transmembrane and intracellular sequences having amino acids 337-405 of SEQ ID NO:2. In some embodiments, the CD28 transmembrane and intracellular sequences comprises amino acids 337-405 of SEQ ID NO:2. In some embodiments, the CD28 transmembrane and intracellular sequences consists essentially of amino acids 337-405 of SEQ ID NO:2. In some embodiments, the CD28 transmembrane and intracellular sequences consists of amino acids 337-405 of SEQ ID NO.2. In some embodiments, the nucleic acid sequence that encodes CD28 transmembrane and intracellular sequences comprises nucleotides 1009-1215 of SEQ ID NO:1. In some embodiments, the nucleic acid sequence that encodes CD28 transmembrane and intracellular sequences consists essentially of nucleotides 1009-1215 of SEQ ID NO.1. In some embodiments, the nucleic acid sequence encodes CD28 transmembrane and intracellular sequences consists of nucleotides 1009-1215 of SEQ ID NO:1.

In some embodiments, CARs comprise 4-1BB intracellular sequences. In some embodiments, CARs comprise 4-1BB intracellular sequences having amino acids 406-444 of SEQ ID NO:2. In some embodiments, CARs comprise 4-1BB intracellular sequences comprise amino acids 406-444 of SEQ ID NO:2. In some embodiments, 4-1BB intracellular sequences consists essentially of amino acids 406-444 of SEQ ID NO:2. In some embodiments, 4-1BB intracellular sequences consist of amino acids 406-444 of SEQ ID NO:2. In some embodiments, the nucleic acid sequence that encodes 4-1BB intracellular comprises nucleotides 1216-1332 of SEQ ID NO: 1. In some embodiments, the nucleic acid sequence that encodes 4-1BB intracellular consists essentially of nucleotides 1216-1332 of SEQ ID NO:1. In some embodiments, the nucleic acid sequence that encodes 4-1BB intracellular consists of nucleotides 1216-1332 of SEQ ID NO:1.

In some embodiments, CARs comprise a sequence encoding at least one immunoreceptor tyrosine activation motif (ITAM). In some embodiments, CARs comprise a sequence from a cell signaling molecule that comprises ITAMs. Typically 3 ITAMS are present in such sequences. Examples of cell signaling molecules that comprise ITAMs include ζ-chain associated with CD3 (CD3ζ), the CD79-alpha and -beta chains of the B cell receptor complex, and certain Fc receptors. Accordingly, in some embodiments, CARs comprise a sequence from a cell signaling molecule such as CD3ζ, the CD79-alpha and -beta chains of the B cell receptor complex, and certain Fc receptors that comprises ITAMs. The sequences included in the CAR are intracellular sequences from such molecules that comprise one of more ITAMs. An ITAM is a conserved sequence of four amino acids that is repeated twice in the cytoplasmic tails of certain cell surface proteins of the immune system. The conserved sequence of four amino sequence of an ITAM contains a tyrosine separated from a leucine or isoleucine by any two other amino acids (YXXL or YXXI which X is independently any amino acid sequence). The ITAM contains at sequence that is typically 14-16 amino acids having the two four amino acid conserved sequences separated by between about 6 and 8 amino acids. The ζ-chain associated with CD3 (CD3ζ) contains 3 ITAMS. Amino acids 445-557 of SEQ ID NO:2 are CD3ζ intracellular sequences. The ITAMS are located at amino acids 465-479, 504-519 and 535-549. In some embodiments, CARs comprise CD3ζ intracellular sequences. In some embodiments, CARs comprise CD3ζ intracellular sequences having amino acids 445-557 of SEQ ID NO:2. In some embodiments, CD3ζ intracellular sequences comprise 445-557 of SEQ ID NO:2. In some embodiments, CD3ζ intracellular sequences consist essentially of 445-557 of SEQ ID NO:2. In some embodiments, CD3ζ intracellular sequences consist of 445-557 of SEQ ID NO:2. In some embodiments, the nucleic and sequence that encodes CD3ζ intracellular comprises nucleotides 333-1671 of SEQ ID NO:1. In some embodiments, the nucleic acid sequence that encodes CD3ζ intracellular consists essentially of nucleotides 1333-1671 of SEQ ID NO:1. In some embodiments, the nucleic acid sequence that encodes CD3ζ intracellular consists of nucleotides 1333-1671 of SEQ ID NO:1.

In some embodiments, CARs may comprise an immunoglobulin-derived antigen binding domain, antibody sequences that bind to GUCY2C fused to a T cell signaling domain such as the CD3zeta signaling chain of the cell receptor or a T-cell costimulatory signaling (e.g. CD28) domain linked to a T-cell chain such as CD3zeta chain or the gamma-signal-transducing subunit of the Ig Fc receptor complex.

The signaling domain of the CAR comprises sequences derived from a TCR. In some embodiments, the CAR comprises an extracellular single chain fragment of antibody variable region that provides antigen binding function fused to a transmembrane and cytoplasmic signaling domain such as CD3zeta chain or CD28 signal domain linked to CD3zeta chain. In some embodiments the signaling domain is linked to the antigen binding domain by a spacer or hinge. When the fragment of antibody variable region binds to GUCY2C, the signaling domain initiates immune cell activation. These recombinant T cells that express membrane bound chimeric receptors comprising an extracellular anti-GUCY2C binding domain and intracellular domain derived from TCRs which perform signaling functions to stimulate lymphocytes. Some embodiments provide anti-GUCY2C binding domain is a single chain variable fragment (scFv) that includes anti-GUCY2C binding regions of the heavy and light chain variable regions of an anti-GUCY2C antibody. A signaling domain may include a T-cell costimulatory signaling (e.g. CD28, 4-1BB (CD137), CD2, CD27, CD30, CD40L, CD79A, CD79B, CD226, DR3, GITR, HVEM, ICOS, LIGHT, OX40, SLAM) domain and T-cell triggering chain (e.g. CD3zeta).

In some embodiments, CARs include an affinity tag. Examples of such affinity tags include: Strep-Tag, Strep-TagII; Poly(His) HA; V5; and FLAG-tag. In some embodiments, the affinity tag may be located before scFv or between scFv and hinge region or after the hinge region. In some embodiments, the affinity tag is selected from Strep-Tag, Strep-TagII, Poly(His), HA; V5, and FLAG-tag, and is located before scFv or between scFv and hinge region or after the hinge region.

In some embodiments, CARs comprise from N terminus to C terminus, a signal sequence, the anti-GCC scFv is a 5F9 single chain variable fragment (scFv), a hinge region, a transmembrane region and intracellular sequences from one of more proteins and intracellular sequences and an immunoreceptor tyrosine activation motif, and optionally an affinity tag.

In some embodiments, CARs comprise from N terminus to C terminus, a signal sequence selected from GM-CSF, CD8 alpha, CD8 beta, CD4, TCR alpha, TCR beta, CD3 delta, CD3 epsilon, CD3 gamma, CD28, BiP linked to the anti-GCC scFv is a 5F9 single chain variable fragment (scFv) selected from (Variable Light Chain fragment—(Glycine₄Serine)₂₋₅ Linker—Variable Heavy Chain fragment) and (Variable Heavy Chain fragment—(Glycine₄Serine)₂₋₅ Linker—Variable Light Chain fragment), linked to a hinge region selected from CD8α, IgG1-Fc and CD28 hinge regions, linked to a transmembrane region selected from a CD8α, IgG1-Fe, IgG4-Fe and CD28 transmembrane region, linked to intracellular sequences selected from CD284-1BB (CD137), CD2, CD27, CD28, CD30, CD40L, CD79A, CD79B, CD226, DR3, GITR, HVEM, ICOS, LIGHT OX40, SLAM intracellular sequences, linked to an immunoreceptor tyrosine activation motif containing sequence selected from CD3ζ, CD79-alpha, CD79-beta and Fc receptor intracellular sequences that comprise one or more ITAMs, optionally linked to an affinity tag selected from Strep-Tag, Strep-TagII, Poly(His), HA; V5, and FLAG-tag.

In some embodiments, CARs comprise from N terminus to C terminus, a Granulocyte-Macrophage Colony-Stimulating Factor (GM-CSF) signal sequence, the anti-GCC scFv is a 5F9 single chain variable fragment (scFv) selected from [Variable Light Chain fragment—(Glycine₄Serine)₂₋₅ Linker—Variable Heavy Chain fragment] or [Variable Heavy Chain fragment—(Glycine₄Serine)₂₋₅ Linker—Variable Light Chain fragment]), a CD8α, CD28, IgG1-Fc, or IgG4-Fc hinge region, a CD8α or CD28 transmembrane and intracellular sequences, 4-1BB intracellular sequences and CD3ζ intracellular sequences.

In some embodiments, CARs consist essentially of a Granulocyte-Macrophage Colony-Stimulating Factor (GM-CSF) signal sequence, the anti-GCC scFv is a 5F9 single chain variable fragment (scFv) (Variable Light fragment—(Glycine₄Serine)₄ Linker—Variable Heavy fragment), as CD8α hinge region, CD28 transmembrane and intracellular sequences, 4-1BB intracellular sequences and CD3ζ intracellular sequences.

In some embodiments, CARs comprise amino acids 1-22, 25-274, 277-336, 337-405, 406-444 and 445-557 of SEQ ID NO:2. In some embodiments, CARs consist essentially of amino acids 1-22, 25-274, 277-336, 337-405, 406-444 and 445-557 of SEQ ID NO:2. In some embodiments, CARs consist of amino acids 1-22, 25-274, 277-336, 337-405, 406-444 and 445-557 of SEQ ID NO:2. In some embodiments, the nucleic acid sequence of the construct that encodes the CARs comprises nucleotides 1-66, 73-822, 829-1008, 1009-1215, 1216-1332 and 1333-1671 of SEQ ID NO:1. In some embodiments, the nucleic acid sequence of the construct that encodes the CARs consist essentially of nucleotides 1-66, 73-822, 829-1008, 1009-1215, 1216-1332 and 1333-1671 of SEQ ID NO:1. In some embodiments, the nucleic acid sequence of the construct that encodes the CARs consist of nucleotides 1-66, 73-822, 829-1008, 1009-1215, 1216-1332 and 1333-1671 of SEQ ID NO:1. In some embodiments, these sequences are linked to regulatory elements necessary for expression of the coding sequence in a human cells such as a human T cell. In some embodiments, a human cell such as a human T cell is transformed with the sequences linked to regulatory elements necessary for expression of the coding sequence.

In some embodiments, the CAR is encoded by GM.5F9(VL-(G4S)4-VH)-CD8a-CD28tm.ICD-4-1BB-CD3z.stop (5F9-28BBz—SEQ ID NO:1), a novel DNA sequence, a synthetic receptor that can be expressed by T lymphocytes and infused for the therapeutic treatment of human guanylyl cyclase C (GUCY2C)-expressing malignancies. GM.5F9(VL-(G4S)4-VH)-CD8a-CD28tm.ICD-4-1BB-CD3z.stop encodes SEQ ID NO:2. 5F9-28BBz comprises human DNA coding sequences concatenated thusly: (1) Granulocyte-Macrophage Colony-Stimulating Factor (GM-CSF) signal sequence, (2) 5F9 single chain variable fragment (scFv) (Variable Light fragment—(Glycine4Serine)4 Linker—Variable Heavy fragment), (3) CD8α hinge region, (4) CD28 transmembrane domain, (5) CD28 intracellular domain, (6) 4-1BB intracellular domain, and (7) CD3ζ intracellular domain. The CAR is referred to as 5F9-28BBz. In some embodiments, the CAR comprises SEQ ID NO:2. In some embodiments, the CAR consists essentially of SEQ ID NO:2. In some embodiments, the CAR consists of SEQ ID NO:2. In some embodiments, the nucleic acid sequence of the construct that encodes the CARs consist of nucleotides comprises SEQ ID NO:1. In some embodiments, the nucleic acid sequence of the construct that encodes the CARs consist of nucleotides consists essentially of SEQ ID NO:1. In some embodiments, the nucleic acid sequence of the construct that encodes the CARs consist of nucleotides consists of SEQ ID NO:1. In some embodiments, these sequences are linked to regulatory elements necessary for expression of the coding sequence in a human cell such as a human cell. In some embodiments, a human cell such as a human T cell transformed with the sequences linked to regulatory elements necessary for expression of the coding sequence.

In some embodiments, the 5F9-28BBz—SEQ ID NO:1 is linked to regulatory elements necessary for expression of the coding sequence in a human cell such as a human T cell. Regulatory elements necessary for expression of the coding sequence in a human cell such as a human T cell may include a promoter, a polyadenylation site and other sequences in 5′ and 3′ untranslated regions. In some embodiments, SEQ ID NO:1 is inserted in an expression vector such as a plasmid such a pVAX, or a retroviral expression sector such as a lentiviral vector, or a recombinant DNA viral vector such a recombinant adenovirus, recombinant AAV, or recombinant vaccinia virus, or as double stranded DNA to be used with CRISPR/Cas9, TALENs, or other transposon technology or as messenger RNA.

In some embodiments, CAR coding sequences are introduced ex vivo into cells, such as T cells, including CD4+ and CD8+, invariant Natural Killer T cells, gamma-delta T cells, Natural Killer cells, and myeloid cells, including CD34+ hematopoietic stem cells from peripheral lymphocytes using routine in vitro gene transfer techniques and materials such as retroviral vectors. Following gene transfer, the recombinant cells are cultured to expand the number of recombinant cells which are administered to a patient. The recombinant cells will recognize and bind to cells displaying the antigen recognized by the extracellular antibody-derived antigen binding domain. Following modification, the cells are expanded ex vivo to obtain large numbers of such cell which are administered to the patient have been described. As above, autologous refers to the donor and recipient of the cells being the same person. Allogenic refers to the donor and recipient of the cells being different people. In addition to isolating and expanding populations of antigen-specific cells by ex vivo culturing, the T cells may be modified after isolating and before expanding populations by having genetic material added to them that encodes proteins such as cytokines, for example IL-2, IL-7, and IL-15.

A plurality of T cells which recognize at least one epitope of GUCY2C may be obtained by isolating a T cell from a cell donor, transforming it with a nucleic acid molecule that encodes an anti-GUCY2C CAR and, culturing the transformed cell to exponentially expand the number of transformed T cells to produce a plurality of such cells.

The cell donor may be the individual to whom the expanded population of cells will be administered, i.e. an autologous cell donor. Alternatively, the T cell may be obtained from a cell donor that is a different individual from the individual to whom the T cells will be administered, i.e. an allogenic T cell. If an allogenic T cell is used, it is preferred that the cell donor be type matched, that is identified as expressing the same or nearly the same set of leukocyte antigens as the recipient.

T cells may be obtained from a cell donor by routine methods including for example, isolation from blood fractions, particularly the peripheral blood monocyte cell component, or from bone marrow samples.

Once T cells are obtained from the cell donor, one or more T cells may be transformed with a nucleic acid that encodes an anti-GUCY2C CAR which includes a functional binding fragment of an antibody that binds to at least one epitope of a GUCY2C and a portion that renders the protein, when expressed in a cell such as a T cell, a membrane bound protein.

The nucleic acid molecule that encodes anti-GUCY2C CAR may be obtained by isolating a B cell that produces antibodies that recognize at least one epitope of GUCY2C from an “antibody gene donor” who has such B cells that produce antibodies that recognizes at least one epitope of GUCY2C. Such antibody gene donors may have B cells that produce antibodies that recognize at least one epitope of a GUCY2C due to an immune response that arises from exposure to an immunogen other than by vaccination or, such antibody gene donors may be identified as those who have received a vaccine which induces production of B cells that produce antibodies that recognize at least one epitope of GUCY2C, i.e. a vaccinated antibody genetic donor. The vaccinated antibody genetic donor may have been previously vaccinated or may be administered a vaccine specifically as part of an effort to generate such B cells that produce antibodies that recognize at least one epitope of GUCY2C for use in a method that comprises transforming T cells with a nucleic acid molecule that encodes an anti-GUCY2C CAR, expanding the cell number, and administering the expanded population of transformed T cells to an individual.

The antibody gene donor may be the individual who will be the recipient of the transformed T cells or a different individual from the individual who will be the recipient of the transformed T cells. The antibody gene donor may be same individual as the cell donor or the antibody gene donor may be a different individual than the cell donor. In some embodiments, the cell donor is the recipient of the transformed T cells and the antibody gene donor is a different individual. In some embodiments, the cell donor is the same individual as the antibody gene donor and is a different individual from the recipient of the transformed T cells. In some embodiments, the cell donor is the same individual as the antibody gene donor and the same individual as the recipient of the transformed T cells.

The nucleic acid molecule which encodes anti-GUCY2C CAR comprises a coding sequence that encodes functional binding fragment of an antibody that recognizes at least one epitope of GUCY2C linked to a protein sequence that provides for the expressed protein to be a membrane bound protein. The coding sequences are linked so that they encode a single product that is expressed.

The coding sequence that encodes a functional binding fragment of an antibody that recognizes at least one epitope of GUCY2C may be isolated from a B cell from an antibody gene donor. Such a B cell may be obtained and the genetic information isolated. In some embodiments, the B cells are used to generate hybrid cells which express the antibody and therefore carry the antibody coding sequence. The antibody coding sequence may be determined, cloned and used to make the abnti-GUCY2C CAR. A functional binding fragment of an antibody that recognizes at least one epitope of GUCY2C may include some or all of the antibody protein which when expressed in the transformed T cells retains its binding activity for at least one epitope of GUCY2C.

The coding sequences for a protein sequence that provides for the expressed protein to be a membrane bound protein may be derived from membrane bound cellular proteins and include the transmembrane domain and, optionally at least a portion of the cytoplasmic domain, and/or a portion of the extracellular domain, and a signal sequence to translocate the expressed protein to the cell membrane.

The nucleic acid molecule that encodes the anti-GUCY2C CAR, i.e, the anti-GUCY2C CAR coding sequence, may be a DNA or RNA The invention relates to chimeric antigen receptors that bind to guanylyl cyclase C and nucleic acid molecules that encode such chimeric antigen receptors. The invention also relates to cells that comprise such chimeric antigen receptors, to methods of making such chimeric antigen receptors and cells, and to methods of using such cells to treat individuals who are suffering from cancer that has cancer cells which express guanylyl cyclase C and to protect individuals against cancer that has cancer cells which express guanylyl cyclase C.

Immunotherapy based upon T cells that express chimeric antigen receptors (CARs) has become an emerging modality for treating cancer. CARs are fusion receptors that comprise a domain which functions to provide HLA-independent binding of cell surface target molecules and a signaling domain that can activate host immune cells of various types, typically peripheral blood T cells, which may include populations of cells referred to cytotoxic lymphocytes, cytotoxic T lymphocytes (CTLs), Natural Killer T cells (NKT) and Natural Killer cells (NK) or helper T cells. That is, while typically being introduced into T cells, genetic material encoding CARs may be added to immune cells that are not T cells such as NK cells.

Guanylyl cyclase C (also referred to interchangeably as GCC or GUCY2C) is a membrane-bound receptor that produces the second messenger cGMP following activation by its hormone ligands guanylin or uroguanylin, regulating intestinal homeostasis, tumorigenesis, and obesity. GUCY2C cell surface expression is confined to luminal surfaces of the intestinal epithelium and a subset of hypothalamic neurons. Its expression is maintained in >95% of colorectal cancer metastases and it is ectopically expressed in tumors that evolve from intestinal metaplasia, including esophageal, gastric, oral, salivary gland and pancreatic cancers.

The inaccessibility of GUCY2C in the apical membranes of polarized epithelial tissue due to subcellular restriction of GUCY2C, creates a therapeutic opportunity to target metastatic lesions of colorectal origin which have lost apical-basolateral polarization, without concomitant intestinal toxicity.

A syngeneic, immunocompetent mouse model demonstrated that CAR-T cells targeting marine GUCY2C were effective against colorectal cancer metastatic to lung in the absence of intestinal toxicities. Similarly, other GUCY2C-targeted therapeutics, including antibody-drug conjugates and vaccines, are safe in preclinical animal models, and therapeutic regimens utilizing these platforms are in clinical trials for metastatic esophageal, gastric, pancreatic, and colorectal cancers (NCT02202759, NCT02202785, NCT01972737).

The safety of these therapeutic regimens, in the context of GUCY2C expression across the rostral-caudal axis of intestine, reflects compartmentalized expression of GUCY2C, enriched in apical, but limited in basolateral, membranes of epithelial cells. Systemic radiolabeled imaging agents conjugated to GUCY2C ligand target GUCY2C-expressing metastases without localizing in intestine, confirming the mucosal compartmentalization of the receptor.

Tumors express up to 10-fold greater amounts of GUCY2C, compared to normal epithelial cells, potentially creating a quantitative therapeutic window to discriminate receptor overexpressing tumors from intestinal epithelium with low/absent GUCY2C in basolateral membranes.

U.S. Patent Application Publication 20120251509 A1 and U.S. Patent Application Publication US 2014-0294784 A1, which are each incorporated herein by reference, disclose CARs including CARs that bind to guanylyl cyclase C, T cells that comprise CARs including T cells that comprise CARs that bind to GUCY2C and target cells that comprise GUCY2C, methods of making chimeric antigen receptors and T cells, and methods of using T cells that comprise CARs that bind to GUCY2C and target cells that comprise GUCY2C to protect individuals against cancer cells that express GUCY2C and to treat individuals who are suffering from cancer in which cancer cells express GUCY2C.

There is remains a need for improved compositions and methods to protect individuals against cancer cells that express GUCY2C and to treat individuals who are suffering from cancer in which cancer cells express GUCY2C.

Proteins comprising an anti-GUCY2C scFV sequence are provided. The anti-GUCY2C scFV sequences may be selected from the group consisting of SEQ ID NO:8, SEQ ID NO:9, SEQ ID NO:10, SEQ ID NO:11, SEQ ID NO:12, SEQ ID NO:13, SEQ ID NO:14 and SEQ ID NO:15.

Proteins comprising the 5F9 anti-GUCY2C scFV sequence and further comprising a signal sequence, a hinge domain, a transmembrane domain, and a signaling domain are provided.

Nucleic acid molecules that encode such proteins are provided. The nucleic acid molecules may be operably linked to regulatory elements that can function to express the protein in a human cell such as a human T cell. The nucleic acid molecules may be incorporated in a nucleic acid vector such as a plasmid or recombinant viral vector that can be used transform human cells into human cells that express the protein.

Human cells comprising the nucleic acid molecules and express the proteins are provided.

Methods of making such cells are provided.

Methods of treating a patient who has cancer that has cancer cells that express GUCY2C and methods of preventing cancer that has cancer cells that express GUCY2C in a patient identified as being of increased risk, are provided.

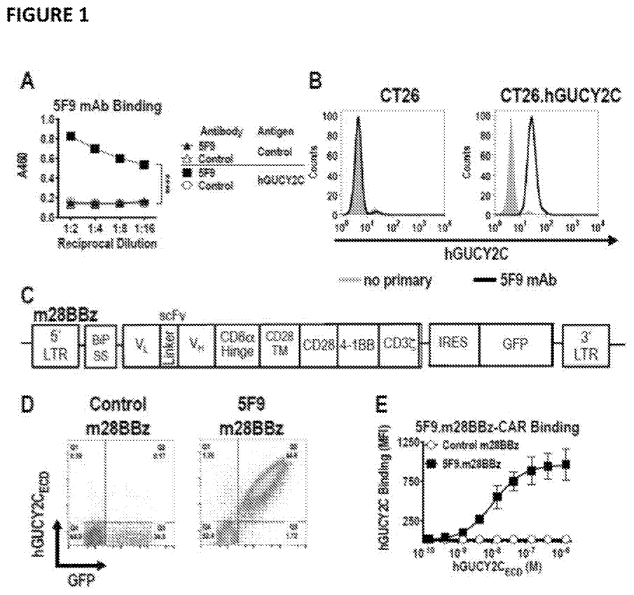

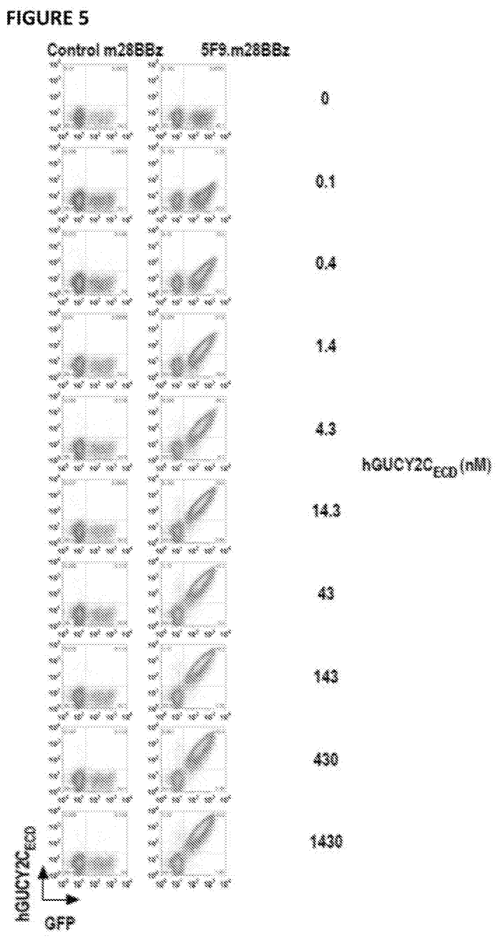

FIG. 1 panels A-E, Generation of human GUCY2C-specific CAR-T cells. (FIG. 1 panel A) Recombinant 5F9 antibody was assessed by ELISA for specific binding to hGUCY2CECD or BSA (negative control) plated at 1 μg/mL. Two-way ANOVA: ****p<0.0001, (FIG. 1 panel B) Flow cytometry analysis was performed on parental CT26 mouse colorectal cancer cells or CT26 cells engineered to express hGUCY2C (CT26.GUCY2C) and stained with 5F9 antibody. (FIG. 1 panel C) Schematic of the third generation murine CAR construct containing murine sequences of the BiP signal sequence, 5F9 scFv, CD8α hinge region, the transmembrane and intracellular domain of CD28, the intracellular domain of 4-1BB (CD137), and the intracellular domain of CD3ζ (5F9.m28BBz). The CAR construct was inserted into the MSCV retroviral plasmid pMIG upstream of an IRES-GFP marker. (FIG. 1 panel D) Murine CD8+ T cells transduced with a retrovirus containing a control (1D3.m28BBz) CAR or CAR derived from the 5F9 antibody (5F9.m28BBz) were labeled with purified 6×His-hGUCY2CECD (10 μg/mL), detected with anti-5×His-Alexa Fluor 647 conjugate. Flow plots were gated on live CD8+ cells. (FIG. 1 panel E) 6×His-hGUCY2CECD binding curves for 5F9-derived or control (1D3) CARs, gated on live CD8+GFP+ cells (See data in FIG. 5). Combined from 3 independent experiments.

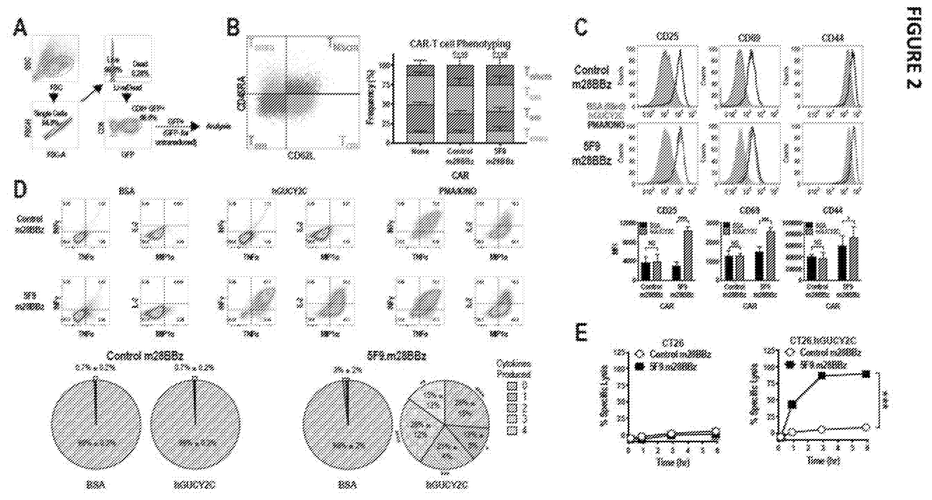

FIG. 2 panels A-E, hGUCY2C-specific CARs mediate antigen-dependent T-cell activation and effector functions. (FIG. 2 panels A-E) Murine CD8+ cells were left non-transduced (None) or transduced with control 1D3.m28BBz or 5F9.m28BBz CAR constructs as indicated. (FIG. 2 panel A) Gating strategy for all analyses in FIG. 2 panels B-D, (FIG. 2 panel B) Representative CAR-T cell phenotyping plot based on CD45RA and CD62L. Two-way ANOVA, NS: not significant; Bars: mean±SD from 2-3 independent experiments; Tn/scm: naïve or T memory stem cells; Tcm: central memory T cells; Tem: effector memory T cells: Temra: effector memory T cells expressing CD45RA. (C-D) 10⁶ CAR-T cells were stimulated for 6 hours with plate-coated antigen (BSA or hGUCY2C) or PMA and ionomycin (PMA/IONO). T-cell activation markers (CD25, CD69, or CD44) and intracellular cytokine production (IFNγ, TNFα, IL2, and MIP1α) were then quantified by flow cytometry. Graphs indicate the mean±SD (FIG. 2 panel C) activation marker upregulation (MFI) and (FIG. 2 panel D) polyfunctional cytokine production (% of CAR+ cells) from 3 independent experiments. (FIG. 2 panel E) Parental CT26 or CT26.hGUCY2C mouse colorectal cancer cells in an E-Plate were treated with CAR-T cells (5:1 E:T ratio), media, or 10% Triton-X 100 (Triton), and the relative electrical impedance was quantified every 15 minutes for 10 hours to quantify cancer cell death (normalized to time=0). Percent specific lysis values were calculated using impedance values following the addition of media and Triton far normalization (0% and 100% specific lysis, respectively). Two-way ANOVA, B-E: *p<0.05, **p<0.01, ***p<0.001, ****p<0.0001.

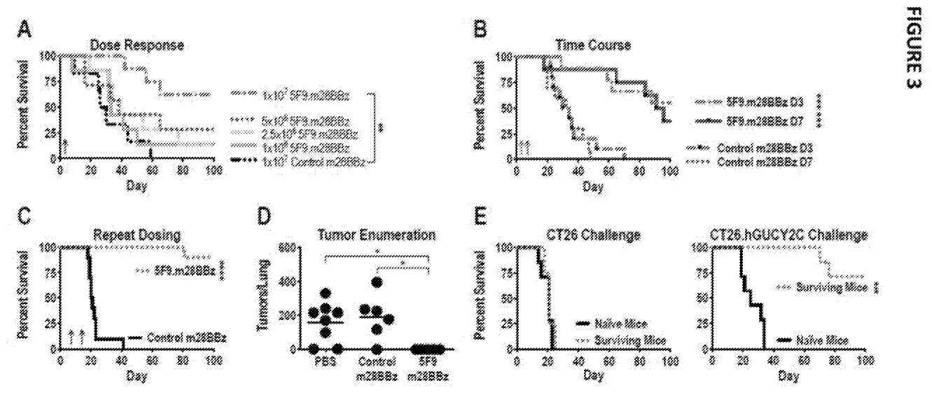

FIG. 3 panels A-E. hGUCY2C CAR-T cells provide long-term protection in a syngeneic lung metastasis model. (FIG. 3 panels A-E) BALB/c mice were injected with 5×10⁵ CT26.hGUCY2C cells via the tail vein to establish lung metastases. Control (4D5.m28BBz) or 5F9.m28BBz CAR constructs were transduced into marine CD8+ T cells. (FIG. 3 panel A) Mice were treated 3 days later with 5 Gy total body irradiation (TBI) followed by 10⁶-10⁷ 5F9.m28BBz (N=7-8/group) or 10⁷ control (N=6) CART cells. (FIG. 3 panel B) Mice were treated on day 3 (D3) or day 7 (D7) with 5 Gy TBI followed by 10⁷ control (N=10/group) or 5F9.m28BBz (N=9-10/group) CAR-T cells. (FIG. 3 panel C) Mire were treated on day 7 with 5 Gy TBI followed by 10⁷ control (N=10) or 5F9.m28BBz (N=12) CART cells on day 7 and day 14. (FIG. 3 panel D) Mice treated on day 7 with 5 Gy TBI and PBS or 10⁷ control or 5F9.m28BBz CAR-T cells were sacrificed on day 18, lungs stained with India ink, and tumors/lung enumerated. One-way ANOVA; *p<0.05, (FIG. 3 panel E) Surviving mice from B and C treated with 5F9.m28BBz CAR-T cells or naïve mice were challenged with 5×10⁵ CT26 (N=4-7/group) or CT26.hGUCY2C (N=7/group) cells (re-challenge occurred 16-40 weeks after initial challenge), Log-rank Mantel-Cox test, FIG. 3 panels A-C and E; **p<0.01, ***p<0.001, ****p<0.0001. Up arrows indicate CAR-T cell treatment days. Each panel indicates an independent experiment.

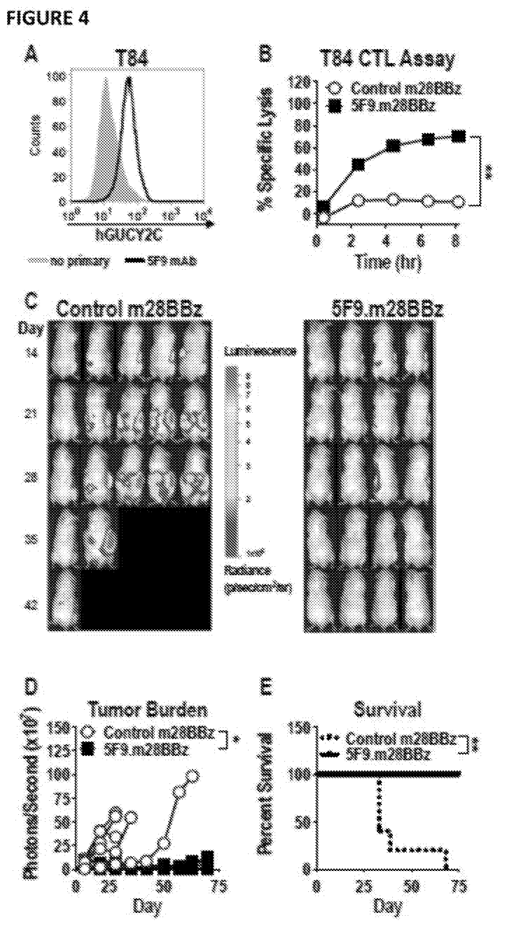

FIG. 4 panels A-E. hGUCY2C CAR-T cells eliminate human colorectal tumor xenografts. (FIG. 4 panel A) hGUCY2C expression on T84 human colorectal cancer cells was quantified by flow cytometry using the recombinant 5F9 antibody. (FIG. 4 panels B-E) Control (1D3.m28BBz) or 5F9.m28BBz CAR constructs were transduced into marine CD8+ T cells. (FIG. 3 panel B) T84 colorectal cancer cells in an E-Plate were treated in duplicate with 5F9-m28BBz or control CAR-T cells (5:1 E:T ratio), media, or 10% Triton-X 100 (Triton), and the relative electrical impedance was measured every 15 minutes for 20 hours to quantify cancer cell death (normalized to time=0). Percent specific lysis values were calculated using impedance values following the addition of media and Triton for normalization (0% and 100% spear specific lysis, respectively). Two-way ANOVA; **p<0.01; representative of two independent experiments. (FIG. 4 panels C-E) Immunodeficient NSG mice were injected with 2.5×10⁶ luciferase-expressing T84 colorectal cancer cells via intraperitoneal injection and were treated with 10⁷ control (N=5) or 5F9-m28BBz (N=4) CAR-T cells on day 14 by intraperitoneal injection. (FIG. 4 panels C-D) Total tumor luminescence (photons/second) was quantified just prior to T-cell injection and weekly thereafter. Two-way ANOVA; *p<0.05. (FIG. 4 panel E) Mice were followed for survival. Log-rank Mantel Cox test; *p<0.05.

FIG. 5. Detection of 5F9.m28BBz CAR surface expression. Murine CD8+ T cells transduced with a retrovirus containing a control m28BBz CAR or CAR derived from the 5F9 antibody (5F9.m28BBz) upstream of an IRES-GFP marker were labeled with purified 6×HishGUCY2CECD (0-1430 mM) and detected with α5×His-Alexa-647 conjugate. Flow plots were gated on live CD8+ cells.

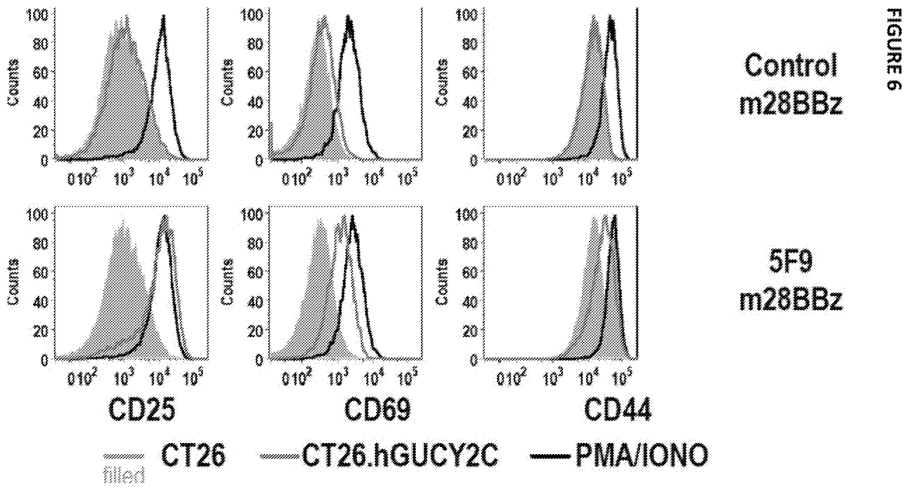

FIG. 6. hGUCY2C-expressing mouse colorectal cancer cells activate 5F9.m28BBz CAR-T cells. 10⁶ CAR-T cells were stimulated for 6 h with 10⁶ parental CT26, CT26.hGUCY2C colorectal cancer cells or PMA and ionomycin (PMA/IONO). T-cell activation markers (CD25, CD69, Of CD44) were quantified by flow cytometry.

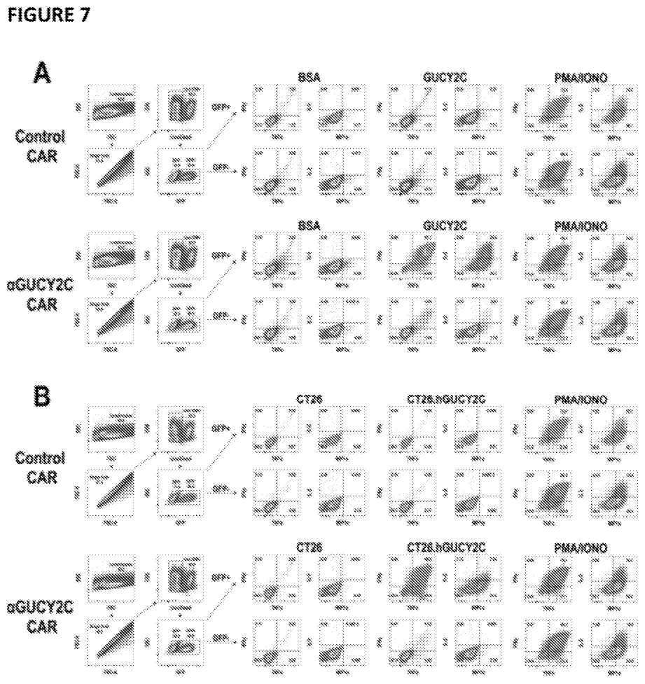

FIG. 7, panels A and B, hGUCY2C-expressing mouse colorectal cancer cells induce 5F9.m28BBz, CAR-T cell cytokine production. 10⁶ CAR-T cells were stimulated for 6 h with plate-coated antigen (FIG. 7, panel A. BSA or hGUCY2C) or 10⁶ parental CT26 or CT26.hGUCY2C colorectal cancer cells (FIG. 7, panel B), or PMA and ionomycin (PMA/IONO). Intracellular cytokine production (IFNγ, TNFα, IL-2 or MIP1α) was quantified by flow cytometry.

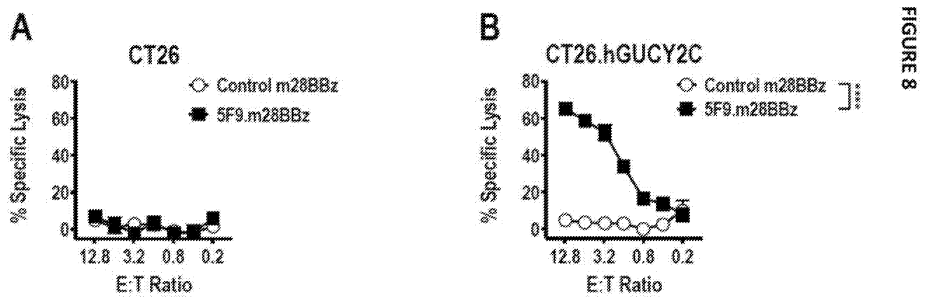

FIG. 8 panels A and B. 5F9.m28BBz CAR-T cells kill hGUCY2C-expressing mouse colorectal cancer cells. β-galactosidase-expressing CT26 or CT26.hGUCY2C mouse colorectal cancer cells were cultured for 4 h with a range of effector CAR-T cell:target cancer cell ratios (E:T Ratio). Specific lysis was determined by β-galactosidase release into the supernatant detected by a luminescent substrate. ****, p<0.0001 (Two-way ANOVA).

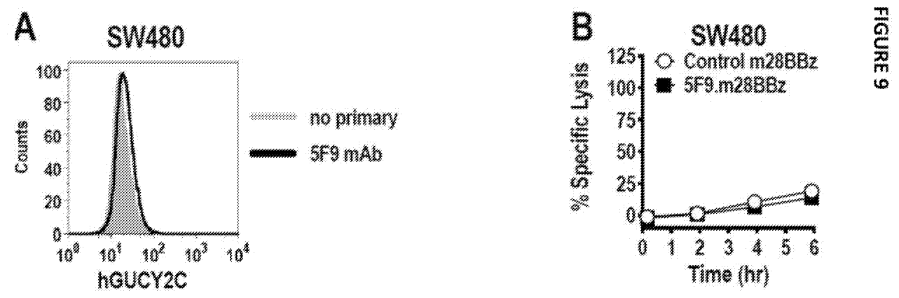

FIG. 9 panels A and B. 5F9.m28BBz CAR-T cells do not kill hGUCY2C-deficient human colorectal tumors. (FIG. 9, panel A) hGUCY2C expression on SW480 human colorectal cancer cells was quantified by flow cytometry using the recombinant 5F9 antibody. (FIG. 9, panel B) SW480 cells in an E-Plate were treated with 5F9.m28BBz or control 1D3.m28BBz CAR T cells, media, or 2.5% Triton-X 100 (Triton) and the relative electrical impedance was quantified every 15 min for 20 h to quantify cancer cell death (normalized to time=0). Percent specific lysis values were calculated using impedance values following the addition of media and Triton for normalization (0% and 100% specific lysis, respectively).

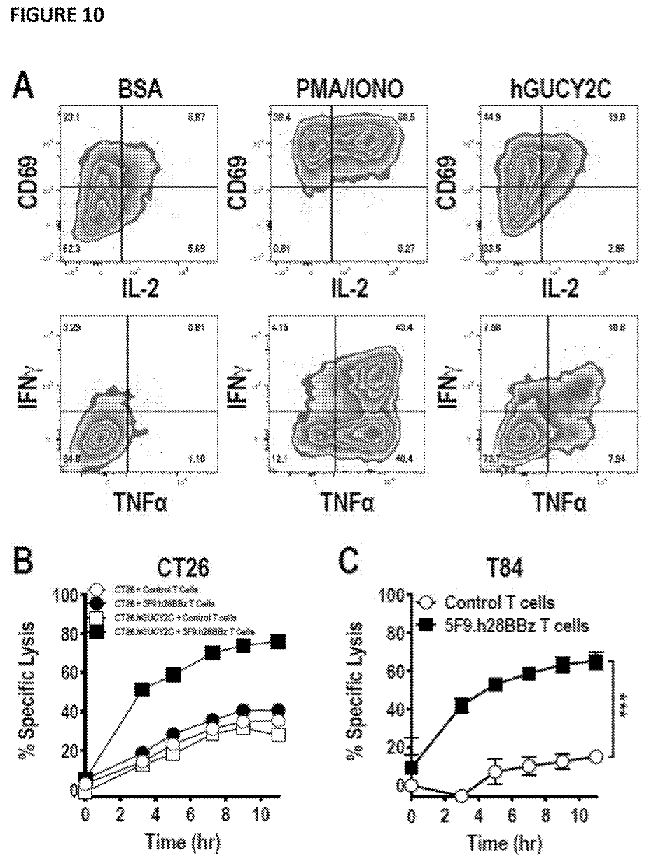

FIG. 10 panels A-C. Human T cells expressing 5F9.h28BBz CAR recognize and kill GUCY2C-expressing colorectal cancer cells. (FIG. 10 panel A) CAR-T cells expressing a human 5F9 CAR construct (5F9.h28BBz) were stimulated for 6 hours with plate-coated antigen (BSA or hGUCY2C) or PMA and ionomycin (PMA/IONO). The T-cell activation marker CD69 and intracellular cytokines (IFNγ, TNFα, and IL-2)□were then quantified by flow cytometry. (FIG. 10 panels B-C) Parental (CT26), human GUCY2Cexpressing CT26 (CT26.hGUCY2C) mouse colorectal cancer cells, (FIG. 10 panel B) or T84 human colorectal cancer cells (FIG. 10 panel C) cultured in an E-Plate were treated with Control or 5F9.h28BBz CAR-T cells (E:T ratio of 10:1), media, or 2.5% Triton-X 100 and the relative electrical impedance was quantified every 15 min to quantify cancer cell death (normalized to time=0). Percent specific lysis values were calculated using impedance values following the addition of media and Triton for normalization (0% and 100% specific lysis, respectively), ***, p<0.001 (Two-way ANOVA).

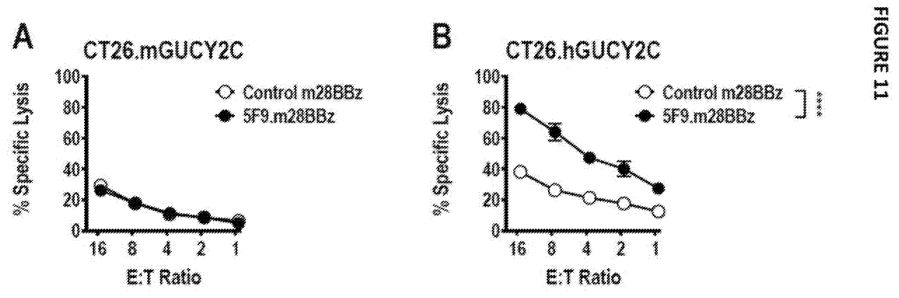

FIG. 11 panels A and B, 5F9.m28BBz CAR-T cells do not kill hGUCY2C-expressing mouse colorectal cancer cells. CT26 cells expressing β-galactosidase and murine GUCY2C (A; CT26.mGUCY2C) or human GUCY2C (B: CT26.hGUCY2C) were cultured for 4 h with a range of effector CAR-T cell:target cancer cell ratios (E:T Ratio). Specific lysis was determined by β-galactosidase release into the supernatant detected by a luminescent substrate, ****, p<0.0001 (Two-way ANOVA).

Single chain protein sequences that bind to the extracellular domain of human GUCY2C were generated using fragments of the variable light chain and variable heavy chain of an anti-GUCY2C antibody that binds to the extracellular domain of human GUCY2C. A linker sequence connects the variable light chain fragment to the variable heavy chain fragment into a single chain antibody variable fragment fusion protein sequence (scFv) that binds to the extracellular domain of human GUCY2C.

The scFv is a component in a CAR, which is a larger fusion protein. The CARs functional components include the immunoglobulin-derived antigen binding domain, antibody sequences i.e, svFv, which binds to human GUCY2C, a hinge domain that links the scFV to a transmembrane domain that anchors the protein in the cell membrane of the cell in which it is expressed, and the signally domain which functions as signal transducing intracellular sequences (also referred to as cytoplasmic sequences) that activate the cell upon scFv binding to human GUCY2C. The nucleic acid sequences that encode the CAR include sequences that encode a signal peptide from a cellular protein that facilitate the transport of the translated CAR to the cell membrane. CARs direct the recombinant cells in which they are expressed to bind to and, in the case of recombinant cytotoxic lymphocytes, recombinant cytotoxic T lymphocytes (CTLs), recombinant Natural Killer T cells (NKT), and recombinant Natural Killer cells (NK) kill cells displaying the antibody-specified target, i.e. GUCY2C. When the CAR is expressed it is transported to the cell surface and the signal peptide is typically removed. The mature CAR functions as a cellular receptor. The scFv and hinge domain are displayed on the cell surface where the scFv sequences can be exposed to proteins on other cells and bind to GUCY2C on such cells. The transmembrance region anchors the CAR in the cell membrane and the intracellular sequences function as a signal domain to transduce a signal in the cell which results in the death of GUCY2C-expressing cell to which the CAR-expressing cell is bound.

In some embodiments, the CARs comprise a signal sequence, such as for example a mammalian or synthetic signal sequence. In some embodiments, the CARs comprise a signal sequence from a membrane-bound protein such as for example a mammalian membrane-bound protein. In some embodiments, the CARs comprise a signal sequence from a membrane-bound protein such as CD8 alpha, CD8 beta, CD4, TCR alpha, TCR beta, CD3 delta, CD3 epsilon, CD3 gamma, CD28, and BiP. Examples of signal sequences may also be found in membrane bound any mammalian signal sequence <http://www.signalpeptide.de/index.php?m-listspdb_mammalia>. In some embodiments, the CARs comprise a Granulocyte-Macrophage Colony-Stimulating Factor (GM-CSF) signal sequence. In some embodiments, the CARs comprise a Granulocyte-Macrophage Colony-Stimulating Factor (GM-CSF) signal sequence having amino acids 1-22 of SEQ ID NO:2. In some embodiments, the Granulocyte-Macrophage Colony-Stimulating Factor (GM-CSF) signal sequence comprises amino acids 1-22 of SEQ ID NO:2. In some embodiments, the Granulocyte-Macrophage Colony-Stimulating Factor (GM-CSF) signal sequence consists essentially of amino acids 1-22 of SEQ ID NO:2. In some embodiments, the Granulocyte-Macrophage Colony-Stimulating Factor (GM-CSF) signal sequence consists of amino acids 1-22 of SEQ ID NO:2. In some embodiments, the nucleic acid sequence of the construct that encodes the CARs that comprise a Granulocyte-Macrophage Colony-Stimulating Factor (GM-CSF) signal sequence comprise nucleic acid 1-66 of SEQ ID NO:1. In some embodiments, the nucleic acid sequence that encodes the Granulocyte Macrophage Colony-Stimulating Factor (GM-CSF) signal sequence comprises nucleic acid 1-66 of SEQ ID NO:1. In some embodiments, the nucleic acid sequence that encodes the Granylocyte-Macrophage Colony-Stimulating Factor (GM-CSF) signal sequence consists essentially of nucleic acid 1-66 of SEQ ID NO:1. In some embodiments, the nucleic acid sequence that encodes the Granulocyte-Macrophage Colony-Stimulating Factor (GM-CSF) signal sequence consists of nucleic acid 1-66 of SEQ ID NO:1.

The anti-GUCY2C binding domain is provided as a single chain chimeric receptor that is MHC-independent. The antigen-binding domain is derived from an antibody. In some embodiments, CARs comprise anti-guanylyl cyclase C (also referred to as GCC or GUCY2C) single chain variable fragment (scFv) (preferably a Variable Light fragment—(Glycine₄Serine)₄ Linker—Variable Heavy fragment) from 5F9. 5F9 is a hybridoma expressing a fully humanized, monoclonal antibody that recognizes the extracellular domain of human GUCY2C. The DNA coding sequences of the antibody heavy and light chains were used to create a novel scFv for CAR implementation that is employed in the creation of anti-GCC CARs, such as for example the 5F9-28BBz CAR, and confers antigen specificity directed towards the GUCY2C molecule.

In some embodiments such as the 5F9-28BBz CAR, the anti-GCC say may be a 5F9 single chain variable fragment (scFv) (Variable Light fragment—(Glycine₄Serine)₄ Linker—Variable Heavy fragment). The 5F9 scFv may comprise amino acids 25-274 of SEQ ID NO:2. In some embodiments, the nucleic acid sequence of the construct that encodes the CARs that comprise the 5F9 scFv comprise nucleotides 73-822 of SEQ ID NO:1. In some embodiments, the CARs comprise an anti-GCC 5F9 scFv. Amino acids 25-133 of SEQ ID NO:2 corresponds to the 5F9 Variable Light chain fragment. Amino acids 154-274 of SEQ ID NO:2 corresponds to the 5F9 Variable Heavy chain fragment. In some embodiments, the CARs comprise an anti-GCC 5F9 single chain variable fragment (scFv) that corresponds to the 5F9 Variable Light fragment and the 5F9 Variable Heavy fragment attached to each other with a (Glycine₄Serine)ₙ LINKER in which (Glycine₄Serine)=GGGGS (SEQ ID NO:3) and n=2-5.

In some embodiments, the linker contains two (Glycine₄Serine) units ((Glycine₄Serine)₂) and may referred to as LINKER G4S-2 (SEQ ID NO:4). In some embodiments, the linker contains three (Glycine₄Serine) units ((Glycine₄Serine)₃) and may referred to as LINKER G4S-3 (SEQ ID NO:5). In some embodiments, the linker contains four (Glycine₄Serine) units ((Glycine₄Serine)₄) and may referred to as LINKER G4S-4 (SEQ ID NO:6). In some embodiments, the linker contains five (Glycine₄Serine) units ((Glycine₄Serine)₅) and may referred to as LINKER G4S-5 (SEQ ID NO:7).

The 5F9 variable fragments may be configured from N-terminus to C-terminus in the order Variable Light Chain fragment-LINKER-Variable Heavy Chain fragment or Variable Heavy Chain fragment-LINKER-Variable Light Chain fragment. In some embodiments, the CARs comprise an anti-GCC 5F9 scFv configured as [5F9 Variable Light Chain fragment—(Glycine₄Serine)₂-5F9 Variable Heavy Chain fragment] (SEQ ID NO:8), [5F9 Variable Light Chain fragment—(Glycine₄Serine)₃-5F9 Variable Heavy Chain fragment] (SEQ ID NO:9), [5E9 Variable Light Chain fragment—(Glycine₄Serine)₄-5F9 Variable Heavy Chain fragment] (SEQ ID NO:10), or [5F9 Variable Light Chain fragment—(Glycine₄Serine)₅-5F9 Variable Heavy Chain fragment] (SEQ ID NO:11). In some embodiments, the CARs comprise an anti-GCC 5F9 scFv configured as [5F9 Variable Heavy Chain fragment—(Glycine₄Serine)₂-5F9 Variable Light Chain fragment] (SEQ ID NO:12), [5F9 Variable Heavy Chain fragment—(Glycine₄Serine)₃-5F9 Variable Light Chain fragment] (SEQ ID NO:13), [5F9 Variable Heavy Chain fragment—(Glycine₄Serine)₄-5F9 Variable Light Chain fragment] (SEQ ID NO:14), or [5F9 Variable Heavy Chain fragment—(Glycine₄Serine)₅-5F9 Variable Light Chain fragment (SEQ ID NO:15).

In some embodiments, the CARs comprise an anti-GCC 5F9 scFv having amino acids 25-274 of SEQ ID NO:2. In some embodiments, the 5F9 say comprises amino acids 25-274 of SEQ ID NO:2. In some embodiments, the 5F9 scFv consists essentially of amino acids 25-274 of SEQ ID NO:2. In some embodiments, the 5F9 scFv consists of amino acids 25-274 of SEQ ID NO:2. In some embodiments, the nucleic acid sequence that encodes the 5F9 scFv comprises nucleotides 73-822 of SEQ ID NO:1. In some embodiments, the nucleic acid sequence that encodes the 5F9 say consists essentially of nucleotides 73-822 of SEQ ID NO:1. In some embodiments, the nucleic acid sequence that encodes the 5F9 scFv consists of nucleotides 73-822 of SEQ ID NO:1.

In some embodiments, CARs comprise a CD8α, IgG1-Fc, IgG4-Fc, or CD28 hinge region. In some embodiments, CARs comprise a CD8α hinge region. In some embodiments, CARs comprise a CD8α hinge region having amino acids 277-336 of SEQ ID NO:2. In some embodiments, the CD8α hinge region composes amino acids 277-336 of SEQ ID NO:2. In some embodiments, the CD8α hinge region consists essentially of amino acids 277-336 of SEQ ID NO:2. In some embodiments, the CD8α hinge region consists of amino acids 277-336 of SEQ ID NO:2. In some embodiments, the nucleic acid sequence that encodes the CD8α hinge region comprises nucleotides 829-1008 of SEQ ID NO:1. In some embodiments, the nucleic acid sequence that encodes the CD8α hinge region consists essentially of nucleotides 829-1008 of SEQ ID NO:1. In some embodiments, the nucleic acid sequence that encodes the CD8α hinge region consists of nucleotides 829-1008 of SEQ ID NO:1.

In some embodiments, CARs comprise a CD28, 4-1BB (CD137), CD2, CD27, CD30, CD40L, CD79A, CD79B, CD226, DR3, GITR, HVEM, ICOS, LIGHT, OX40, or SLAM transmembrane region.

In some embodiments, CARs comprise a CD28, 4-1BB (CD137), CD2, CD27, CD30, CD40L, CD79A, CD79B, CD226, DR3, GITR, HVEM, ICOS, LIGHT, OX40, or SLAM intracellular region.

In some embodiments, CARs comprise both transmembrane and intracellular (cytoplasmic) sequences from CD28, 4-1BB (CD137), CD2, CD27, CD30, CD40L, CD79A, CD79B, CD226, DR3, GITR, HVEM, ICOS, LIGHT, OX40, or SLAM. In some embodiments, CARs comprise CD28 transmembrane and intracellular sequences. In some embodiments, CARs comprise CD28 transmembrane and intracellular sequences having amino acids 337-405 of SEQ ID NO:2. In some embodiments, the CD28 transmembrane and intracellular sequences comprises amino acids 337-405 of SEQ ID NO:2. In some embodiments, the CD28 transmembrane and intracellular sequences consists essentially of amino acids 337-405 of SEQ ID NO:2. In some embodiments, the CD28 transmembrane and intracellular sequences consists of amino acids 337-405 of SEQ ID NO:2. In some embodiments, the nucleic acid sequence that encodes CD28 transmembrane and intracellular sequences comprises nucleotides 1009-1215 of SEQ ID NO:1. In some embodiments, the nucleic acid sequence that encodes CD28 transmembrane and intracellular sequences consists essentially of nucleotides 1009-1215 of SEQ ID NO:1. In some embodiments, the nucleic acid sequence encodes CD28 transmembrane and intracellular sequences consists of nucleotides 1009-1215 of SEQ ID NO:1.

In some embodiments, CARs comprise intracellular (cytoplasmic) sequences from ζ-chain associated with CD3 (CD3ζ), the CD79-alpha and -beta chains of the B cell receptor complex, or certain Fc receptors.

In some embodiments, CARs comprise a) intracellular (cytoplasmic) sequences from one or more of CD28, 4-1BB (CD137), CD2, CD27, CD30, CD40L, CD79A, CD79B, CD226, DR3, GITR, HVEM, ICOS, LIGHT, OX40, or SLAM intracellular region in combination with b) intracellular (cytoplasmic) sequences from ζ-chain associated with CD3 (CD3ζ), the CD79-alpha and -beta chains of the B cell receptor complex, or certain Fc receptors.

In some embodiments, CARs comprise CD28 transmembrane and intracellular sequences together with 4-1BB intracellular sequences in combination with CD3ζ intracellular sequences.

In some embodiments, CARs comprise CD28 transmembrane and intracellular sequences having amino acids 337-405 of SEQ ID NO:2. In some embodiments, the CD28 transmembrane and intracellular sequences comprises amino acids 337-405 of SEQ ID NO:2. In some embodiments, the CD28 transmembrane and intracellular sequences consists essentially of amino acids 337-405 of SEQ ID NO:2. In some embodiments, the CD28 transmembrane and intracellular sequences consists of amino acids 337-405 of SEQ ID NO:2. In some embodiments, the nucleic acid sequence that encodes CD28 transmembrane and intracellular sequences comprises nucleotides 1009-1215 of SEQ ID NO:1. In some embodiments, the nucleic acid sequence that encodes CD28 transmembrane and intracellular sequences consists essentially of nucleotides 1009-1215 of SEQ ID NO:1. In some embodiments, the nucleic acid sequence encodes CD28 transmembrane and intracellular sequences consists of nucleotides 1009-1215 of SEQ ID NO:1.

In some embodiments, CARs comprise 4-1BB intracellular sequences. In some embodiments, CARs comprise 4-1BB intracellular sequences having amino acids 406-444 of SEQ ID NO:2. In some embodiments, CARs comprise 4-1BB intracellular sequences comprise amino acids 406-444 of SEQ ID NO:2. In some embodiments, 4-1BB intracellular sequences consists essentially of amino acids 406-444 of SEQ ID NO:2. In some embodiments, 4-1BB intracellular sequences consist of amino acids 406-444 of SEQ ID NO:2. In some embodiments, the nucleic acid sequence that encodes 4-1BB intracellular comprises nucleotides 1216-1332 of SEQ ID NO:1. In some embodiments, the nucleic acid sequence that encodes 4-1BB intracellular consists essentially of nucleotides 1216-1332 of SEQ ID NO:1. In some embodiments, the nucleic acid sequence that encodes 4-1BB intracellular consists of nucleotides 1216-1332 of SEQ ID NO:1.

In some embodiments, CARs comprise a sequence encoding at least one immunoreceptor tyrosine activation motif (ITAM). In some embodiments, CARs comprise a sequence from a cell signaling molecule that comprises ITAMs. Typically 3 ITAMS are present in such sequences. Examples of cell signaling molecules that comprise ITAMs include ζ-chain associated with CD3 (CD3ζ), the CD79-alpha and -beta chains of the B cell receptor complex, and certain Fc receptors. Accordingly, in some embodiments, CARs comprise a sequence from a cell signaling molecule such as CD3ζ, the CD79-alpha and -beta chains of the B cell receptor complex, and certain Fc receptors that comprises ITAMs. The sequences included in the CAR are intracellular sequences from such molecules that comprise one of more ITAMs. An ITAM is a conserved sequence of four amino acids that is repeated twice in the cytoplasmic tails of certain cell surface proteins of the immune system. The conserved sequence of four amino sequence of an ITAM contains a tyrosine separated from a leucine or isoleucine by any two other amino acids (YXXL or YXXI in which X is independently any amino acid sequence). The ITAM contains a sequence that is typically 14-16 amino acids having the two four amino acid conserved sequences separated by between about 6 and 8 amino acids. The ζ-chain associated with CD3 (CD3ζ) contains 3 ITAMS. Amino acids 445-557 of SEQ ID NO:2 are CD3ζ intracellular sequences. The ITAMS are located at amino acids 465-479, 504-519 and 535-549. In some embodiments, CARs comprise CD3ζ intracellular sequences. In some embodiments, CARs comprise CD3ζ intracellular sequences having amino acids 445-557 of SEQ ID NO:2. In some embodiments, CD3ζ intracellular sequences comprise 445-557 of SEQ ID NO:2. In some embodiments, CD3ζ intracellular sequences consist essentially of 445-557 of SEQ ID NO:2. In some embodiments, CD3ζ intracellular sequences consist of 445-557 of SEQ ID NO:2. In some embodiments, the nucleic acid sequence that encodes CD3ζ intracellular comprises nucleotides 1333-1671 of SEQ ID NO:1. In some embodiments, the nucleic acid sequence that encodes CD3ζ intracellular consists essentially of nucleotides 1333-1671 of SEQ ID NO:1. In some embodiments, the nucleic acid sequence that encodes CD3ζ intracellular consists of nucleotides 1333-1671 of SEQ ID NO:1.

In some embodiments, CARs may comprise an immunoglobulin-derived antigen binding domain, antibody sequences that bind to GUCY2C fused to a T cell signaling domain such as the CD3zeta signaling chain of the cell receptor or a T-cell costimulatory signaling (e.g. CD28) domain linked to a T-cell chain such as CD3zeta chain or the gamma-signal-transducing subunit of the Ig Fc receptor complex.

The signaling domain of the CAR comprises sequences derived from a TCR. In some embodiments, the CAR comprises an extracellular single chain fragment of antibody variable region that provides antigen binding function fused to a transmembrane and cytoplasmic signaling domain such as CD3zeta chain or CD28 signal domain linked to CD3zeta chain. In some embodiments the signaling domain is linked to the antigen binding domain by a spacer or hinge. When the fragment of antibody variable region binds to GUCY2C, the signaling domain initiates immune cell activation. These recombinant T cells that express membrane bound chimeric receptors comprising an extracellular anti-GUCY2C binding domain and intracellular domain derived from TCRs which perform signaling functions to stimulate lymphocytes. Some embodiments provide anti-GUCY2C binding domain is a single chain variable fragment (scFv) that includes anti-GUCY2C binding regions of the heavy and light chain variable regions of an anti-GUCY2C antibody. A signaling domain may include a T-cell costimulatory signaling (e.g. CD28, 4-1BB (CD137), CD2, CD27, CD 30, CD40L, CD79A, CD79B, CD226, DR3, GITR, HVEM, ICOS, LIGHT, OX40, SLAM) domain and T-cell triggering chain (e.g. CD3zeta).

In some embodiments, CARs include an affinity tag. Examples of such affinity tags include: Strep-Tag; Strep-TagII; Poly(His); HA; V5; and FLAG-tag. In some embodiments, the affinity tag may be located before scFv or between scFv and hinge region or after the hinge region. In some embodiments, the affinity tag is selected from Strep-Tag, Strep-TagII, Poly(His), HA; V5, and FLAG-tag, and is located before scFv or between scFv and hinge region or after the hinge region.

In some embodiments, CARs comprise from N terminus to C terminus, a signal sequence, the anti-GCC scFv is a 5F9 single chain variable fragment (scFv), a hinge region, a transmembrane region and intracellular sequences from one of more proteins and intracellular sequences and an immunoreceptor tyrosine activation motif, and optionally an affinity tag.

In some embodiments, CARs comprise from N terminus to C terminus, a signal sequence selected from GM-CSF, CD8 alpha, CD8 beta, CD4, TCR alpha, TCR beta, CD3 delta, CD3 epsilon, CD3 gamma, CD28, BiP linked to the anti-GCC scFv is a 5F9 single chain variable fragment (scFv) selected from (Variable Light Chain fragment—(Glycine₄Serine)₂₋₅ Linker—Variable Heavy Chain fragment) and (Variable Heavy Chain fragment—(Glycine₄Serine)₂₋₅ Linker—Variable Light Chain fragment), linked to a hinge region selected from CD8α, IgG1-Fc, IgG4-Fc and CD28 hinge regions, linked to a transmembrane region selected from a CD8α, IgG1-Fc, IgG1-Fc and CD28 transmembrane region, linked to intracellular sequences selected from CD284-1BB (CD137), CD2, CD27, CD28, CD30, CD40L, CD79A, CD79B, CD226, DR3, GITR, HVEM, ICOS, LIGHT, OX40, SLAM intracellular sequences, linked to an immunoreceptor tyrosine activation motif containing sequence selected from CD3ζ, CD79-alpha, CD79-beta and Fc receptor intracellular sequences that comprise one or more ITAMs, optionally linked to an affinity of tag selected from Strep-Tag, Strep-TagII, Poly(His), HA; V5, and FLAG-tag.

In some embodiments, CARs comprise from N terminus to C terminus, a Granulocyte-Macrophage Colony-Stimulating Factor (GM-CSF) signal sequence, the anti-GCC scFv is a 5F9 single chain variable fragment (scFv) selected from [Variable Light Chain fragment—(Glycine₁Serine)₂₋₅ Linker—Variable Heavy Chain fragment] or [Variable Heavy Chain fragment—(Glycine₄Serine)₂₋₅ Linker—Variable Light Chain frament]), a CD8α, CD28, IgG1-Fc, or IgG4-Fc hinge region, a CD8α or CD28 transmembrane and intracellular sequences, 4-1BB intracellular sequences and CD3ζ intracellular sequences.

In some embodiments, CARs consist essentially of a Granulocyte-Macrophage Colony-Stimulating Factor (GM-CSF) signal sequence, the anti-GCC scFv is a 5F9 single chain variable fragment (scFv) (Variable Light fragment—(Glycine₄Serine)₄ Linker—Variable Heavy fragment), a CD8α hinge region, CD28 transmembrane and intracellular sequences, 4-1BB intracellular sequences and CD3ζ intracellular sequences.

In some embodiments, CARs comprise amino acids 1-22, 25-274, 277-336, 337-405, 406-444 and 445-557 of SEQ ID NO:2. In some embodiments, CARs consist essentially of amino acids 1-22, 25-274, 277-336, 337-405, 406-444 and 145-557 of SEQ ID NO:2. In some embodiments, CARs consist of amino acids 1-22, 25-274, 277-336, 337-405, 406-444 and 445-557 of SEQ ID NO:2. In some embodiments, the nucleic acid sequence of the construct that encodes the CARs comprises nucleotides 1-66, 73-822, 829-1008, 1009-1215, 1216-1332 and 1333-1671 of SEQ ID NO:1. In some embodiments, the nucleic acid sequence of the construct that encodes the CARs consist essentially of nucleotides 1-66, 73-822, 829-1008, 1009-1215, 1216-1332 and 1333-1671 of SEQ ID NO:1. In some embodiments, the nucleic acid sequence of the construct that encodes the CARs consist of nucleotides 1-66, 73-822, 829-1008, 1009-1215, 1216-1332 and 1333-1671 of SEQ ID NO:1. In some embodiments, these sequences are linked to regulatory elements necessary for expression of the coding sequence in a human cells such as a human T cell. In some embodiments, a human cell such as a human T cell is transformed with the sequences linked to regulatory elements necessary for expression of the coding sequence.

In some embodiments, the CAR is encoded by GM.5F9(VL-(G4S)4-VH)-CD8a-CD28tm.ICD-4-1BB-CD3z.stop (5F9-28BBz—SEQ ID NO:1), a novel DNA sequence, a synthetic receptor that can be expressed by T lymphocytes and infused for the therapeutic treatment of human guanylylcyclase C (GUCY2C)-expressing malignancies. GM.5F9(VL-(G4S)4-VH)-CD8a-CD28tm.ICD-4-1BB-CD3z.stop encodes SEQ ID NO:2. 5F9-28BBz comprises human DNA coding sequences concatenated thusly: (1) Granulocyte-Macrophage Colony-Stimulating Factor (GM-CSF) signal sequence, (2) 5F9 single chain variable fragment (scFv) (Variable Light fragment—(Glycine4Serine)4 Linker—Variable Heavy fragment), (3) CD8α hinge region, (4) CD28 transmembrane domain, (5) CD28 intracellular domain, (6) 4-1BB intracellular domain, and (7) CD3ζ intracellular domain. The CAR is referred to as 5F9-28BBz. In some embodiments, the CAR comprises SEQ ID NO:2. In some embodiments, the CAR consists essentially of SEQ ID NO:2. In some embodiments, the CAR consists of SEQ ID NO:2. In some embodiments, the nucleic acid sequence of the construct that encodes the CARs consist of nucleotides comprises SEQ ID NO:1. In some embodiments, the nucleic acid sequence of the construct that encodes the CARs consist of nucleotides consists essentially of SEQ ID NO:1. In some embodiments, the nucleic acid sequence of the construct that encodes the CARs consist of nucleotides consists of SEQ ID NO:1. In some embodiments, these sequences are linked to regulatory elements necessary for expression of the coding sequence in a human cell such as a human T cell. In some embodiments, a human cell such as a human T cell transformed with the sequences linked to regulatory elements necessary for expression of the coding sequence.

In some embodiments, the 5F9-28BBz—SEQ ID NO:1 is linked to regulatory elements necessary for expression of the coding sequence in a human cell such as a human T cell. Regulatory elements necessary for expression of the coding sequence in a human cell such as a human T cell may include a promoter, a polyadenylation site and other sequences in 5′ and 3′ untranslated regions. In some embodiments, SEQ ID NO:1 is inserted in an expression vector such as a plasmid such a pVAX, or a retroviral expression vector such as a lentiviral vector, or a recombinant DNA viral vector such a recombinant adenovirus, recombinant AAV, or recombinant vaccinia virus, or as double stranded DNA to be used with CRISPR/Cas9, TALENs, or other transposon technology or as messenger RNA.

In some embodiments, CAR coding sequences are introduced ex vivo into cells, such as T cells, including CD4+ and CD8+, invariant Natural Killer T cells, gamma-delta T cells, Natural Killer cells, and myeloid cells, including CD34+ hematopoietic stem cells from peripheral lymphocytes using routine in vitro gene transfer techniques and materials such as retroviral vectors. Following gene transfer, the recombinant cells are cultured to expand the number of recombinant cells which are administered to a patient. The recombinant cells will recognize and bind to cells displaying the antigen recognized by the extracellular antibody-derived antigen binding domain. Following modification, the cells are expanded ex vivo to obtain large numbers of such cell which are administered to the patient have been described. As above, autologous refers to the donor and recipient of the cells being the same person. Allogenic refers to the donor and recipient of the cells being different people. In addition to isolating and expanding populations of antigen-specific T cells by ex vivo culturing, the T cells may be modified after isolating and before expanding populations by having genetic material added to them that encodes proteins such as cytokines, for example IL-2, IL-7, and IL-15.

A plurality of T cells which recognize at least one epitope of GUCY2C may be obtained by isolating a T cell from a cell donor, transforming it with a nucleic acid molecule that encodes an anti-GUCY2C CAR and, culturing the transformed cell to exponentially expand the number of transformed T cells to produce a plurality of such cells.

The cell donor may be the individual to whom the expanded population of cells will be administered, i.e. an autologous cell donor. Alternatively, the T cell may be obtained from a cell donor that is a different individual from the individual to whom the T cells will be administered, i.e. an allogenic T cell. If an allogenic cell is used, it is preferred that the cell donor be type matched, that is identified as expressing the same or nearly the same set of leukocyte antigens as the recipient.

T cells may be obtained from a cell donor by routine methods including, for example, isolation from blood fractions, particularly the peripheral blood monocyte cell component, or from bone marrow samples.

Once T cells are obtained from the cell donor, one or more T cells may be transformed with a nucleic acid that encodes an anti-GUCY2C CAR which includes a functional binding fragment of an antibody that binds to at least one epitope of a GUCY2C and a portion that renders the protein, when expressed in a cell such as a T cell, a membrane bound protein.

The nucleic acid molecule that encodes anti-GUCY2C CAR may be obtained by isolating a B cell that produces antibodies that recognize at least one epitope of GUCY2C from an “antibody gene donor” who has such B cells that produce antibodies that recognizes at least one epitope of GUCY2C. Such antibody gene donors may have B cells that produce antibodies that recognize at least one epitope of a GUCY2C due to an immune response that arises from exposure to an immunogen other than by vaccination or, such antibody gene donors may be identified as those who have received a vaccine which induces production of B cells that produce antibodies that recognize at least one epitope of GUCY2C, i.e, a vaccinated antibody genetic donor. The vaccinated antibody genetic donor may have been previously vaccinated or may be administered a vaccine specifically as part of an effort to generate such B cells that produce antibodies that recognize at least one epitope of GUCY2C for use in a method that comprises transforming T cells with a nucleic acid molecule that encodes an anti-GUCY2C CAR, expanding the cell number, and administering the expanded population of transformed T cells to an individual.

The antibody gene donor may be the individual who will be the recipient of the transformed T cells or a different individual from the individual who will be the recipient of the transformed T cells. The antibody gene donor may be same individual as the cell donor or the antibody gene donor may be a different individual than the cell donor. In some embodiments, the cell donor is the recipient of the transformed T cells and the antibody gene donor is a different individual. In some embodiments, the cell donor is the same individual as the antibody gene donor and is a different individual from the recipient of the transformed T cells. In some embodiments, the cell donor is the same individual as the antibody gene donor and the same individual as the recipient of the transformed cells.

The nucleic acid molecule which encodes anti-GUCY2C CAR comprises a coding sequence that encodes functional binding fragment of an antibody that recognizes at least one epitope of GUCY2C linked to a protein sequence that provides for the expressed protein to be a membrane bound protein. The coding sequences are linked so that they encode a single product that is expressed.

The coding sequence that encodes a functional binding fragment of an antibody that recognizes at least one epitope of GUCY2C may be isolated from a B cell from an antibody gene donor. Such a B cell may be obtained and the genetic information isolated. In some embodiments, the B cells are used to generate hybrid cells which express the antibody and therefore carry the antibody coding sequence. The antibody coding sequence may be determined, cloned and used to make the abnti-GUCY2C CAR. A functional binding fragment of an antibody that recognizes at least one epitope of GUCY2C may include some or all of the antibody protein which when expressed in the transformed T cells retains its binding activity for at least one epitope of GUCY2C.

The coding sequences for a protein sequence that provides for the expressed protein to be a membrane bound protein may be derived from membrane bound cellular proteins and include the transmembrane domain and, optionally at least a portion of the cytoplasmic domain, and/or a portion of the extracellular domain, and a signal sequence to translocate the expressed protein to the cell membrane.

molecule. The nucleic acid molecule may be operably linked to the regulatory elements necessary for expression of the coding sequence in a donor T cell. In some embodiments, the nucleic acid molecule that comprises an anti-GUCY2C CAR coding sequence is a plasmid DNA molecule. In some embodiments, the nucleic acid molecule that comprises an anti-GUCY2C CAR coding sequence is a plasmid DNA molecule that is an expression vector wherein the coding sequence is operably linked to the regulatory elements in the plasmid that are necessary for expression of the anti-GUCY2C CAR coding sequence in a donor T cell. In some embodiments, a nucleic acid molecule that comprises an anti-GUCY2C CAR coding sequence may be incorporated into viral particle which is used to infect a donor T cell. Packaging technology for preparing such particles is known. The coding sequence incorporated into the particle may be operable linked to regulatory elements in the plasmid that are necessary for expression of the anti-GUCY2C CAR coding sequence in a donor T cell. In some embodiments, the nucleic acid molecule that comprises an anti-GUCY2C CAR coding sequence is incorporated into a viral genome. In some embodiments, the viral genome is incorporated into viral particle which is used to infect a donor T cell. Viral vectors for delivery nucleic acid molecules to cells are well known and include, for example, viral vectors based upon vaccine virus, adenovirus, adeno associated virus, pox virus as well as various retroviruses. The anti-GUCY2C CAR coding sequence incorporated into the viral genome may be operable linked to regulatory elements in the plasmid that are necessary for expression of the anti-GUCY2C CAR coding sequence in a donor T cell.

Upon expression of the nucleic acid in the transformed T cells, the transformed cells may be tested to identify a T cell that recognizes at least one epitope of GUCY2C. Such transformed T cells may be identified and isolated from the sample using standard techniques. The protein that comprises at least one epitope of GUCY2C may be adhered to a solid support and contacted with the sample. T cells that remain on the surface after washing are then further tested to identify T cells that which recognize at least one epitope of GUCY2C. Affinity isolation methods such as columns, labeled protein that binds to the cells, cell sorter technology may also be variously employed. T cells that recognize at least one epitope of GUCY2C may also be identified by their reactivity in the presence of a protein with at least one epitope of GUCY2C.

Once a T cell is identified as a T cell that recognizes at least one epitope GUCY2C, it may be clonally expanded using tissue culture techniques with conditions that promote and maintain cell growth and division to produce an exponential number of identical cells. The expanded population of T cells may be collected for administration to a patient.

A plurality of T cells that recognize at least an epitope of GUCY2C according to some embodiments comprise a pharmaceutically acceptable carrier in combination with the cells. Pharmaceutical formulations comprising cells are well known and may be routinely formulated by one having ordinary skill in the art. Suitable pharmaceutical carriers are described in Remington's Pharmaceutical Sciences, A. Osol, a standard reference text in this field, which is incorporated herein by reference. The present invention relates to pharmaceutical composition for infusion.

In some embodiments, for example, the plurality of cells can be formulated as a suspension in association with a pharmaceutically acceptable vehicle. Examples of such vehicles are water, saline, Ringer's solution, dextrose solution, and 5% human serum albumin. The vehicle may contain additives that maintain isotonicity (e.g., sodium chloride, mannitol) and chemical stability (e.g., buffers and preservatives). The vehicle is sterilized prior to addition of cells by commonly used techniques.

The plurality of cells may be administered by any means that enables them to come into contact with cancer cells. Pharmaceutical compositions may be administered intravenously for example.

Dosage varies depending upon the nature of the plurality of cells, the age, health, and weight of the recipient; nature and extent of symptoms, kind of concurrent treatment, frequency of treatment, and the effect desired. Generally, 1×10¹⁰ to 1×10¹² cells are administered although more or fewer may also be administered, such as 1×10⁹ to 1×10¹³. Typically, 1×1011 T cells are administered. The number of cells delivered is the amount sufficient to induce a protective or therapeutically response. Those having ordinary skill in the art can readily determine the range and optimal dosage by routine methods.

Patients to be treated with the anti-GUCY2C CARs include patients who have cancer cells that express GUCY2C. In some embodiments, such cancers may be metastatic colorectal cancer, metastatic or primary stomach, metastatic or primary esophageal, metastatic or primary oral, metastatic or primary salivary gland or metastatic or primary pancreatic cancer or any other cancer identified as having GUCY2C expression. In some embodiments, patients suspected of having cancer that includes cancer cells which express GUCY2C are treated with anti-GUCY2C CARs. In some embodiments, prior to treatment with anti-GUCY2C CARs, patients are identified as metastatic colorectal cancer, metastatic or primary stomach, metastatic or primary esophageal, metastatic or primary oral, metastatic or primary salivary gland or metastatic or primary pancreatic cancer patients. In some embodiments, prior to treatment with anti-GUCY2C CARs, samples of cancer from a patient is tested for GUCY2C expression and those patients with cancers that test positive for GUCY2C expression are treated with anti-GUCY2C CARs. In some embodiments, prior to treatment with anti-GUCY2C CARs, a patient undergoes surgery to remove a tumor and a sample of the tumor removed from the patient is tested for GUCY2C expression and those patients with cancers that test positive for GUCY2C expression are treated with anti-GUCY2C CARs.

The anti-GUCY2C CARs may be useful to prevent cancer in individuals identified at being at an elevated risk of cancer that has cancer cells that express GUCY2C such as metastatic colorectal cancer, metastatic or primary stomach, metastatic or primary esophageal, metastatic or primary oral, metastatic or primary salivary gland or metastatic or primary pancreatic cancer. An individual may be identified at being at an elevated risk of cancer that has cancer cells that express GUCY2C based upon family medical history, genetic background or prior diagnosis of cancer that has cancer cells that express GUCY2C such as metastatic colorectal cancer, metastatic or primary stomach, metastatic or primary esophageal, metastatic or primary oral, metastatic or primary salivary gland or metastatic or primary pancreatic cancer and treatment removing the cancer or treatment resulting in apparent remission or cancer free status.

### EXAMPLES

### Example 1

A human GUCY2C-targeted CAR that may be employed in patients with GUCY2C-expressing malignancies such as metastatic colorectal cancer, metastatic or primary stomach, esophageal, oral, salivary gland or pancreatic cancer or other cancers that express GUCY2C has been identified. This anti-GUCY2C CAR induced antigen-dependent T-cell activation, cytokine production, and cytolytic activity. Human GUCY2C-targeted CAR-T cells were effective against metastatic tumors in immunocompetent, syngeneic mouse models, as well as enograft models of human colorectal cancer.

### Materials and Methods

Cell lines and reagents. CT26 and β-galactosidase—expressing CT26.CL25 mouse colorectal cancer cell lines and the human colorectal cancer cell lines T84 and SW480 were obtained from ATCC and large stocks of low-passage cells were cryopreserved. Cells were authenticated by the original suppliers and routinely authenticated by morphology, growth, antibiotic resistance (where appropriate GUCY2C and β-galactosidase expression, and pattern of metastasis in vivo and routinely screened for mycoplasma using the Universal Mycoplasma Detection Kit (ATCC, Cat. No. 30-1012K). Before injection into mice, cells were routinely cultured for <2weeks. The gene encoding human GUCY2C was codon-optimized and synthesized (Gene Art, Life Technologies) and cloned into the retroviral construct pMSCVpuro (Clontech). CT26.hGUCY2C and CT26.CL25.hGUCY2C were generated by transducing CT26 and CT26.CL25 cells with retroviral supernatants encoding hGUCY2C, followed by selection with puromycin. Retroviral supernatants ere produced by transfecting the Phoenix-Eco retroviral packaging cell line (Gary Nolan, Stanford University) with pMSCV-Puro (Clontech) or hGUCY2C-pMSCV Pure and the pCL-Eco (Imgenex) retroviral packaging vector (12). Luciferase containing T84.fLuc cells were generated by transduction with lentiviral supernatants generated by transfecting 293FT cells (Invitrogen) with pLenti4-V5-GW-Iuciferase.puro (kindly provided by Andrew Aplin, Thomas Jefferson University) and the ViraPower Lentiviral Packaging Mix (Invitrogen) according in manufacturer instructions, followed by selection in puromycin. The single chain variable fragment (scFv) from the human GUCY2C-specific antibody 5F9 was cloned into the pFUSE-rIgG-Fc2 (IL2ss) plasmid (Invivogen), producing a 5F9 scFv fusion protein with rabbit Fc (5F9-rFc), 5F9-rFc was collected in supernatants of transfected 293F cells (Life Technologies), titrated in ELISA plates (Nunc-Immuno PolySorp) coated with BSA or recombinant 6×His-tagged hGUCY2C extracellular domain (6×His-hGUCY2CECD) protein purified under contract from HEK293-6E cells by GenScript and detected with HRP-conjugated goat anti-rabbit (Jackson ImmunoResearch). For flow cytometry, cells were stained with 5F9-rFc or control supernatants from untransfected 293F cells diluted in FACS buffer (1% heat-inactivated PBS in PBS), followed by secondary Alexa Fluor 488-conjugated anti-rabbit (Life Technologies) in FACS buffer. Cells were fixed with 2% paraformaldehyde (PFA: Affymetrix) and analyzed using the BD LSR II flow cytometer and FlowJo v10 software (Tree Star).

Murine CAR-T Generation. Murine CAR components were employed to produce a third-generation, codon-optimized retroviral CAR construct as previously described. A codon-optimized scFv sequence derived from the 5F9 human GUCY2C-specific antibody was cloned into a CAR construct containing murine sequences of the BiP signal peptide, CD8α hinge region, CD28 transmembrane and intracellular domains, and 4-1BB (CD137) and CD3ζ intracellular domains, producing the 5F9.m28BBz CAR construct. CARs derived from the human ERBB2 (Her2)-specific antibody 4D5 or mouse CD19-specific antibody 1D3 were used as controls as indicated (Control m28BBz). CARs were subcloned into the pMSCV-IRES-GFP (pMIG) retroviral vector (Addgene #27490). The Phoenix-Eco retroviral packaging cell line (Gary Nolan, Stanford University) was transfected with CAR-pMIG vectors and the pCL-Eco retroviral packaging vector (Imgenex) using the Calcium Phosphate Profection^{R} Mammalian Transfection System (Promega). Retrovirus-containing supernatants were collected 48 hours later, filtered through 0.45 μM filters, and aliquots were frozen at −80° C. Murine CD8+ T cells were negatively selected from BALB/c splenocytes using the CD8α+ T cell Isolation Kit II and LS magnetic columns (Miltenyi Biotec). CD8⁺ T cells were subsequently stimulated with anti-CD3/anti-CD28-coated beads (T Cell Activation/Expansion Kit, Miltenyi Biotec) at a 1:1 bead:cell ratio at 1×10⁶ cells/ml in cRPMI with 100 U/mL recombinant human IL2 (NCI Repository). The day following stimulation, ½ of the culture media was carefully replaced with an equal volume of thawed retroviral supernatant in the presence of 8 μg/ml polybrene (Millipore). Spinoculation was performed at room temperature for 90 minutes at 2500 rpm followed by incubation at 37° C. for 2.5 hours at which point cells were pelleted and resuspended in fresh media containing 100 U/mL IL2. T cells were expanded for 7-10 days by daily dilution to 1×10⁶ cells/ml with fresh cRPMI and IL2 at which point they were used for functional assays,

Human CAR-T Cell Generation. For studies with human T cells, PBMCs were collected from consenting volunteers in accordance with regulatory and institutional requirements. MACS (Stemcell Technologies) purified CD8⁺ T cells were negatively selected from individual normal healthy donor whole blood at >90% purity. CAR domains employing human sequences were used to produce a third-generation, codon-optimized retroviral CAR construct containing the 5F9 human GUCY2C-specific scFv and human sequences of the GM-CSF signal peptide, CD8α hinge region, CD28 transmembrane and intracellular domains, and 4-1BB (CD137) and CD3ζ intracellular domains producing 5F9.h28BBz (SEQ 10 NO:1). CAR-encoding amphotropic γ-166 retrovirus production was similar to that with murine T cells, but replaced pCL-Eco with the pCL-Ampho packaging plasmid (Imgenex). Retroviral transduction occurred on day 3 or 4 post-activation with ImmunoCult CD3/CD28 Activator (Stem Cell Technologies). Cells underwent flow sorting for GFP-enrichment on day 7, followed by experimental use on day 10. Throughout the duration in culture, human CD8+ T cells were maintained in ImmunoCult-XF media (Stemcell Technologies) supplemented with 100 U/mL recombinant, human IL2 (NCI Repository).

CAR Surface Detection. CAR-transduced T cells were stained with the LIVE/DEAD Fixable Aqua Dead Cell Stain kit (Invitrogen) in PBS, labeled with varying concentrations of 6×His-hGUCY2CECD for 1 hour in PBS 0.5% BSA, stained with anti-5×His-Alexa Fluor 647 conjugate (Qiagen) and anti-CD8b-PE (clone H35.17.2, BD Biosciences) for 1 hour in PBS 0.5% BSA, fixed with 2% PFA and analyzed using the BD LSR II flow cytometer and FlowJo software v10 (Tree Star). hGUCY2C binding was determined by mean fluorescence intensity of Alexa Fluor 647 on live CD8+ CAR+ (GFP+) cells. Non-linear regression analysis (GraphPad Prism v6) was used to determine the Kav and Bmax of GUCY2C-CAR binding.

Mouse T-cell Phenotyping, Activation Markers, and Intracellular Cytokine Staining. For phenotyping, 1×10⁶ non-transduced or CAR-transduced mouse T cells were stained with LIVE/DEAD Fixable Aqua Dead Cell Stain kit (Invitrogen) in PBS and subsequently stained for surface markers using anti-CD8α-BV570 (clone RPA-T8; Biolegend), anti-CD45RA—PerCP-Cy5.5 (clone 14.8; BD Biosciences), and anti-CD62L-PE-Cy7 (clone MEL-14; BD Biosciences) for 30 minutes in PBS 0.5% BSA. Cells were subsequently fixed and permeabilized (BD Cytofix/Cytoperm Kit; BD Biosciences) with Cytofix/Cytoperm buffer for 20 minutes at 4° C. and stained for intracellular GFP (anti-GFP-Alexa Fluor 488; Invitrogen) for 45 minutes in Perm/Wash buffer to identify CAR-transduced cells. The following subsets were then quantified based on CD45RA and CD62L staining: Tn/scm (naïve or T memory stem cells; CD62L+CD45RA+), Tcm (central memory T cells; CD62L+CD45RA−), Tem (effector memory T cells; CD62L CD45RA−), and Temra (effector memory T cells expressing CD45RA; CD62L CD45RA+). For activation marker and cytokine analysis, 1×10⁶ CAR-transduced mouse T cells were stimulated for 6 hours in tissue culture plates previously coated with 1 μg/mL, GUCY2C in PBS overnight at 4° C. or in tissue culture plates containing 1×10⁶ CT26 or CT26.hGUCY2C cells. As a positive control, CAR-T cells were incubated for 6 hours with 1× Cell Stimulation Cocktail (PMA/Ionomycin, eBioscience). Incubation included 1× Protein Transport Inhibitor Cocktail (eBioscience) when assessing intracellular cytokines. Cells were stained with LIVE/DEAD Fixable Aqua Dead Cell Stain kit (Invitrogen) and subsequently stained for surface markers using anti-CD8α-PerCP-Cy5.5 (clone 53.6-7; BD Biosciences), anti-CD69-PE (clone H1.2F3; BD Biosciences), anti-CD25-PE (clone PC61.5, eBioscience), and anti-CD44-APC (clone IM7; Biolegend). Intracellular cytokine staining was performed using the BD Cytofix/Cytoperm Kit (BD Biosciences) and staining with anti-GFP-Alexa488 (Invitrogen), anti-IFNγ-APC-Cy7 (clone XMG1.2; BD Biosciences), anti-TNFα-PE-Cy7 (clone MP6-XT22; BD Biosciences), anti-IL2-APC (clone JES6-5H4; BD Biosciences) and αMIP1α-PE (clone 39624; R&D Systems). Cells were fixed in 2% PFA and analyzed on a BD LSR II flow cytometer. Analyses were performed using FlowJo v10 software (Tree Star).

Human T-cell Activation Marker and Intracellular Cytokine Staining. For activation marker and cytokine analysis, 1×10⁶ human GUCY2C-directed CAR transduced human T cells were stimulated for 6 hours in tissue culture plates coated overnight at 4° C. with 10 human GUCY2C or BSA control antigen in PBS or with 1× Cell Stimulation Cocktail (PMA/Ionomycin, eBioscience) added at the time of plating CAR-T cells. All conditions included 1× Protein Transport Inhibitor Cocktail (eBioscience) at the beginning of the incubation period. Cells were stained with LIVE/DEAD Fixable Aqua Dead Cell Stain kit (lnvitrogen) in PBS for 10 minutes and subsequently stained for surface markers using anti-CD8-Qdot 800 (clone 3B5, Invitrogen) and anti-CD69-APC (clone L78, BD Biosciences) in PBS 0.5% BSA for 25 minutes. Intracellular cytokine staining was performed using the BD Cytofix/Cytoperm Kit (BD Biosciences) consisting of fixation with Cytofix/Cytoperm buffer for 20 minutes and staining with anti-GFP-Alexa Fluor 488 (Invitrogen), anti-IFNγ-BV605 (clone 4S.B3; BioLegend), anti-TNFα-PerCP-Cy5.5 (clone Mab11; BD Biosciences), and anti-IL2-PE (clone MQ1-17H12; BD Biosciences) in BD perm wash buffer for 45 minutes. Cells were fixed in 2% PFA and analyzed on a BD LSR II flow cytometer. Analyses were performed using FlowJo v10 software (Tree Star).

T-Cell Cytotoxicity Assays. The xCELLigence real-time, cell-mediated cytotoxicity system (Area Biosciences Inc.) was utilized for assessment of CAR T cell-mediated cytotoxicity as previously described (12). Briefly, 1×104 CT26 or CT26.hGUCY2C or 2.5×104 T84 or SW480 cancer cell targets were plated in 150 μL of growth medium in each well of an E-Plate 16 (Area Biosciences) and grown overnight in a 37° C. incubator, quantifying electrical impedance every 15 minutes using the RTCA DP Analyzer system and RTCA software version 2.0 (Area Biosciences Inc.). Approximately 24 hours later for mouse and 6 hours for human T cell experiments, 50 μL of CAR-T cells were added (5:1 E:T ratio for mouse T cells or 10:1 E:T ratio for human T cells), or 50 μL of media or 10% Triton-X 100 (Fisher) was added for a final (v/v) of 2.5% Triton-X 100 as negative and positive controls, respectively. Cell-mediated killing was quantified over the next 10-20 hours, reading electrical impedance every 15 minutes. Percent specific lysis values were calculated using GraphPad Prism Software v6 for each replicate at each time point, using impedance values following the addition of media and Triton-X 100 for normalization (0% and 100% specific lysis, respectively). Alternatively, the β-gal release T-cell cytotoxicity assay utilized CT26 cancer cell targets expressing β-galactosidase (CT26.CL25). Cancer cell targets were plated at 2×10⁵ cells/well in a 96-well plate and incubated with increasing effector CAR T cell to cancer cell target ratios for 4 hours at 37° C. Released β-galactosidase was measured in the media using the Galacto-Light Plus System (Applied Biosystems, Carlsbad, Calif). Maximum release was determined from supernatants of cells that were lysed with supplied lysis buffer. 258% specific lysis=[(experimental release−spontaneous release)/(maximum release−spontaneous release)]×100.

Metastatic Tumor Models. BALB/c mice and NSG (NOD-scid IL2Rγnull) mice were obtained from the NCI Animal Production Program (Frederick, Md.) and Jackson Labs (Bar Harbor, Me.), respectively. Animal protocols were approved by the Thomas Jefferson University Institutional Animal Care and Use Committee. In syngeneic mouse models, BALB/c mice were injected with 5×10⁵ CT26.hGUCY2C cells in 100 μL of PBS by tail vein injection to establish lung metastases. On indicated days, mice received a non-myeloablative dose of 5 Gy total body irradiation in a PanTak, 310 kVe x-ray machine. Mice received the indicated dose of CAR-T cells produced from CD8⁺ BALB/c T cells in 100 μL of PBS by tail vein at the indicated time points. Mice were followed for survival or sacrificed on day 18 after cancer cell injection and lungs were stained with India Ink and fixed in Fekete's solution for tumor enumeration. For re-challenge experiments, naïve mice or mice cleared of established tumors by CAR-T cells (referred to as “surviving mice”) received one dose of 5×10⁵ CT26 or CT26.hGUCY2C via tail vein injection. Surviving mice were initially challenged 16-40 weeks prior to the re-challenge experiment. In human tumor xenograft models, NSG (23) mice (JAX stock #005557) were injected with 2.5×10⁶ T84.fLuc cells in 100 μL PBS via intraperitoneal injection. Mice received a dose of 10⁷ total (not sorted on CAR⁺) T cells produced from CD8⁺ BALB/c T cells in 100 μL PBS via intraperitoneal injection on day 14 after cancer cell inoculation. Tumor growth was monitored 281 weekly by subcutaneous injection of a 250 μL solution of 15 mg/ml D-luciferin potassium salt (Gold Biotechnologies) in PBS and imaging after 8 minutes of exposure using the Caliper IVIS Lumina-XR imaging station (Perkin Elmer). Total radiance (photons/second) was quantified using Living Image In Vivo Imaging Software (Perkin Elmer).

### Results and Discussion

hGUCY2C CAR-T Cells

A recombinant antibody (clone 5F9) specific for human GUCY2C (hGUCY2C) bound to purified hGUCY2C extracellular domain (FIG. 1 panel A) and murine CT26 colorectal cancer cells engineered to express hGUCY2C, but not hGUCY2C deficient CT26 cancer cells (FIG. 1 panel B). The 5F9 scFv was used to generate a third-generation murine CAR construct (5F9.m28BBz) containing the BiP signal sequence, CD8α hinge region, and intracellular CD28, 4-1BB, and CD3ζ signaling moieties and inserted into a retroviral construct (FIG. 1 panel C). Retroviruses encoding control m28BBz or 5F9.m28BBz CARs were used to transduce murine T cells with ˜35-45% transduction efficiency, quantified by a GFP transduction marker (FIG. 1 panel D). hGUCY2C-binding avidity (Kav=11.2 nM) and CAR expression (Bmax=957.8 MFI), quantified by incubating CAR-T cells with increasing concentrations of purified 6×His-tagged hGUCY2CECD followed by detection with labeled α5×His antibody and assessment by flow cytometry, was comparable to CARs that exhibited functional reactivity to mouse GUCY2C (12) ((FIG. 1 panels D-E and SEQ ID NO:1).

hGUCY2C CAR Mediates T-Cell Activation and Effector Function

Transduction of purified mouse CD8⁺ T cells with control m28BBz or hGUCY2C specific 5F9.m28BBz CAR constructs had no impact on T-cell phenotype compared to non-transduced cells (FIG. 2 panel B), producing a mixture of memory and effector phenotypes [Tn/scm (CD62L+CD45RA+), Tcm (CD62L+CD45RA−), Tem (CD62L-CD45RA−) and Temra (CD62L-CD45RA+)] similar to other CAR constructs in CAR-T cells produced in the presence of IL1. hGUCY2C-specific, but not control, CAR-T cells upregulated the activation markers CD25, CD69, and CD44 (FIG. 2 panel C) and produced the effector cytokines IFNγ, TNFα, IL2, and MIP1α (FIG. 2 panel D) when stimulated with immobilized hGUCY2CECD protein or CT26.hGUCY2C cells (FIG. 6 and FIG. 7 panels A and B). Activation marker and cytokine responses were absent when 5F9.m28BBz CAR-T cells were stimulated with BSA or hGUCY2C-deficient CT26 cells, confirming that T-cell activation by the 5F9.m28BBz CAR is antigen-dependent ((FIG. 2 panels C-D, FIG. 6 and FIG. 7 panels A and B). Although 5F9.m28BBz CAR-T cells were inactive against hGUCY2C deficient CT26 cells in vitro (FIG. 2 panel E), they exhibited time-dependent killing of CT26.hGUCY2C cells, quantified by employing an electrical impedance assay (FIG. 2 panel E) and confirmed in a β-galactosidase release T-cell cytotoxicity assay (FIG. 8 panels A and B).

hGUCY2C CAR-T Cells Oppose Metastatic Colorectal Cancer

The endogenous immune system can induce immunosuppression in the tumor microenvironment and compete with adoptively transferred T cells for resources necessary for long-term persistence. In that context, lympho-depletive conditioning regimens, such as low-dose total body irradiation (TBI) or chemotherapies, enhance the efficacy of adoptively transferred T cells by eliminating immunosuppressive cells and reducing competition for homeostatic cytokines, including IL7 and IL15. An immunocompetent mouse model and a non-myeloablative dose of 5 Gy total body irradiation (TBI) was utilized to mimic clinical treatment regimens. Immunocompetent BALB/c mice received CT26.hGUCY2C cells by tail vein to produce lung metastases, followed 3 days later by TBI and increasing doses of mouse CAR-T cells (FIG. 3 panel A). hGUCY2C targeted 5F9.m28BBz, but not control, CAR-T cells improved survival of mice at a dose of 10⁷ T cells (FIG. 3 panel A). This dose also was effective when administered 7 days after cancer cell inoculation (FIG. 3 panel B), and a second dose administered on day 14 further increased median survival compared to a single dose on day 7 (>150 vs 93.5 days, p<0.05; FIG. 3 panel C). Lungs collected 18 days after cancer cell inoculation (11 days after treatment) contained tumor modules, confirming that control mice succumbed to lung metastases while 5F9.m28BBz CAR-T cell treatment eliminated macroscopic tumors (FIG. 3 panel D). To determine if surviving mice exhibited persistent protection against hGUCY2C-expressing tumors, long term survivors (161-282 days following initial cancer cell inoculation) were challenged with either parental CT26 or CT26.hGUCY2C cells by tail vein injection to examine hGUCY2C-specific protection (FIG. 3 panel E). CT26 minors are known to harbor the gp70 envelope protein derived from murine leukemia virus that generates protective gp70-specific CD8+ T-cell responses in some vaccination regimens. Long-term surviving and naïve mice challenged with parental CT26 cancer cells exhibited identical death rates, indicating that long-term survivors did not produce a protective immune response to gp70 or other antigens expressed in CT26 cells (FIG. 3 panel E). Conversely, long-term survivors were protected against CT26.hGUCY2C cancer cells compared to naïve control mice, indicating that 5F9.m28BBz CAR-T cells produce persistent protection against hGUCY2C-expressing tumors (FIG. 3 panel E).

hGUCY2C CAR-T Cells Recognize Human Colorectal Tumors

Next, it was determined if hGUCY2C CAR-T cells recognized native hGUCY2C or human colorectal tumors. The recombinant hGUCY2C-specific antibody 5F9 stained hGUCY2C on the surface of GUCY2C-expressing T84 (FIG. 4 panel A), but not GUCY2C-deficient SW480 (FIG. 9 panel A), human colorectal cancer cells. Correspondingly, 5F9.m28BBz CAR-T cells lysed T84 (FIG. 4 panel B), but not SW480 (FIG. 9 panel B), cancer cells in vitro in a time-dependent manner. Control CAR-T cells did not kill either human cancer cell type, indicating that killing was antigen-dependent (FIG. 4 panel A and FIG. 9 panel A). Human T cells expressing a human 5F9 CAR construct (5F9.h28BBz) produced effector cytokines following GUCY2C stimulation and killed human colorectal cancer cells endogenously expressing hGUCY2C (FIG. 10 panels A-C). Mouse T cells expressing hGUCY2C-specific (5F9.m28BBz), but not control, CAR effectively treated T84 human colorectal tumor xenografts in a peritoneal metastases model (FIG. 4 panels C-E). Together, these data indicated that hGUCY2Cspecific CAR constructs produced with the 5F9 scFv can redirect T cell mediated killing of human colorectal tumors endogenously expressing hGUCY2C.

Adoptive T-cell therapies targeting colorectal tumor antigens have been limited by antigen “on-target, off-tumor” toxicities. GUCY2C were previously validated as a potential target for CAR-T cell treatment in a completely syngeneic mouse model in which CARs targeting mouse GUCY2C promoted antitumor efficacy in the absence of toxicities to the normal GUCY2C-expressing intestinal epithelium. Here, a human GUCY2C-specific CAR was produced from an antibody that is currently employed as an antibody-drug conjugate in clinical trials for GUCY2C-expressing malignancies (NCT02202759, NCT02202785) and demonstrated its ability to induce T-cell activation, effector function, and antitumor efficacy in both syngeneic and human colorectal tumor xenograft mouse models using murine T cells. CARs produced from the 5F9 scFv do not cross-react with murine GUCY2C (FIG. 11 panels A and B), preventing quantification of intestinal toxicity in mouse models. Differences in the antigen-recognition domain of the CAR described here and the murine CAR previously described, as well as inherent differences between mice and humans, suggest caution in GUCY2C CAR-T cell administration to humans, despite murine GUCY2C CAR-T cell safety data. Thus, appropriate safety measures should be considered when translating the use of GUCY2C CAR-T cells into the clinic, including transient CAR expression by mRNA electroporation or incorporation of suicide genes. Nevertheless, GUCY2C-targeted CAR-T cells are an attractive tool for the T-cell therapy armamentarium, a paradigm that is limited by the lack of suitable antigen targets. Following further development in human T-cell systems and translation to human clinical trials, GUCY2C CAR-T cell therapy may potentially transform treatment of metastatic gastrointestinal malignancies, a disease setting with limited therapeutic options that produces >140,000 deaths annually in the US.

### Example 2

Transfer may be combined with various treatments including cytokine administration (primarily IL-2), CMA-directed vaccination and of antibody therapy, chemotherapy, host preparative lymphodepletion with cyclophosphamide and fludarabine total-body irradiation (TBI), among other potential adjunct treatments.

### REFERENCES

Maude S L, Frey N, Shaw P A, Aplenc R, Barrett D M, Bunin N J, et al. Chimeric antigen receptor T cells for sustained remissions in leukemia, N Engl J Med 2014; 371(16):1507-17 doi 10.1056/NEJMoa1407222.

Lee D W, Kochenderfer J N, Stetler-Stevenson M, Cui Y K, Delbrook C, Feldman S A, et al. T cells expressing CD19 chimeric antigen receptors for acute lymphoblastic leukaemia in children and young adults: a phase 1 dose-escalation trial. Lancet 2015; 385(9967):517-28 doi 10.1016/S0140-6736(14)61403-3.

Louis C U, Savoldo B, Dotti G, Pule M, Yvon E, Myers G D, et al. Antitumor activity and long-term fate of chimeric antigen receptor-positive T cells in patients with neuroblastoma. Blood 2011; 118(23):6050-6 doi 10.1182/blood-2011-05-354449.

Parkhurst M R, Yang J C, Langan R C, Dudley M E, Nathan D A, Feldman S A, et al. T cells targeting carcinoembryonic antigen can mediate regression of metastatic colorectal cancer but induce severe transient colitis. Mol Ther 2011; 19(3):620-6 doi 10.1038/mt.2010.272.

Morgan R A, Yang J C, Kitano M, Dudley M E, Laurencot C M, Rosenberg S A. Case report of a serious adverse event following the administration of T cells transduced with a chimeric antigen receptor recognizing ERBB2. Mol Ther 2010; 18(4):843-51 doi mt201024 [pii] 10.1038/mt.2010.24.

Aka A A, Rappaport J A, Pattison A M, Sato T, Snook A E, Waldman S A. Guanylate cyclase C as a target for prevention, detection, and therapy in colorectal cancer. Expert review of clinical pharmacology 2017; 10(5):549-57 doi 10.1080/17512433.2017.1292124.

Valentino M A, Lin J E, Snook A E, Li P, Kim G W, Marszalowicz G. et al. A uroguanylin-GUCY2C endocrine axis regulates feeding in mice, J Clin Invest 2011; 121(9):3578-88 doi 10.1172/JCI57925.

Carrithers S L, Barber M T, Biswas S, Parkinson S J, Park P K, Goldstein S D, et al. Guanylyl cyclase C is a selective marker for metastatic colorectal tumors in human extraintestinal tissues. Proc Natl Acad Sci USA 1996; 93(25):14827-32.

Birbe R, Palazzo J P, Walters R, Weinberg D, Schulz S, Waldman S A. Guanylyl cyclase C is a marker of intestinal metaplasia, dysplasia, and adenocarcinoma of the gastrointestinal tract. Hum Pathol 2005; 36(2):170-9 doi 10.1016/j.humpath.2004.12.002.

Wolfe H R, Mendizabal M. Lleong F, Cuthbertson A, Desai V, Pullan S, et al. In vivo imaging of human colon cancer xenografts in immunodeficient mice using a guanylyl cyclase C—specific ligand. J Nucl Med 2002; 43(3):392-9.

Coradini D, Casarsa C, Oriana S. Epithelial cell polarity and tumorigenesis: new perspectives for cancer detection and treatment. Acta Pharmacol Sin 2011; 32(5):552-64 doi 10.1038/aps.2011.20.

Magee M S, Kraft C L, Abraham T S, Baybutt T R, Marszalowicz G P, Li P, et al. GUCY2C-directed CAR-T cells oppose colorectal cancer metastases without autoimmunity. Oncoimmunology 2016; 5(10):e1227897 doi 10.1080/2162402x.2016.1227897.

Marszalowicz G P, Snook A E, Magee M S, Merlino D, Berman-Booty L D, Waldman S A. GUCY2C lysosomotropic endocytosis delivers immunotoxin therapy to metastatic colorectal cancer. Oncotarget 2014; 5(19);9460-71doi 10.18632/oncotarget.2455.

Snook A E, Li P, Stafford B J, Faul E J, Huang L. Birbe R C, et al. Lineage specific T-cell responses to cancer mucosa antigen oppose systemic metastases without mucosal inflammatory disease. Cancer Res 2009; 69(8):3537-44 doi 10.1158/0008-5472.CAN-08-3386.

Snook A E, Magee M S, Schulz S, Waldman S A. Selective antigen-specific CD4(+) T-cell, but not CD8(+) T- or B-cell, tolerance corrupts cancer immunotherapy. Eur J Immunol 2014; 44(7): 1956-66 doi 10.1002/eji.201444539.

Hodson C A, Ambrogi I G, Scott R O, Mohler P J, Milgram S L, Polarized apical sorting of guanylyl cyclase C is specified by a cytosolic signal. Traffic 2006; 7(4);456-64 doi TRA398 [pii] 10.1111/j.1600-0854.2006.00398.x.

Charney A N, Egnor R W, Alexander-Chacko J T, Zaharia V, Mann E A, Giannella R A. Effect of E. coli heat-stable enterotoxin on colonic transport in guanylyl cyclase C receptor-deficient mice. Am J Physiol Gastrointest Liver Physiol 2001; 280(2):G216-21.

Kuhn M, Adermann K, Jahne J, Forssmann W G, Rechkemmer G. Segmental differences in the effects of guanylin and Escherichia coli heat stable enterotoxin on Cl-secretion in human gut. The Journal of Physiology 1994; 479 (Pt 3):433-40.

Guarino A, Cohen M B, Overmann G, Thompson M R, Giannella R A. Binding of E. coli heat-stable enterotoxin to rat intestinal brush borders and to basolateral membranes, Dig Dis Sci 1987; 32(9):1017-26.

Witek M E, Nielsen K, Walters R, Hyslop T, Palazzo J, Schulz S, et al. The putative tumor suppressor Cdx2 is overexpressed by colorectal adenocarcinomas. Clin Cancer Res 2005; 11(24 Pt 1):8549-56 doi 10.1158/1078-0432.CCR-05-1624.

Liu X, Jiang S, Fang C, Yang S, Olalere D, Pequignot E C, et al. Affinity-Tuned ErbB2 or EGFR Chimeric Antigen Receptor T Cells Exhibit an Increased Therapeutic Index against Tumors in Mice. Cancer Res 2015; 75(17):3596-607 doi 10.1158/0008-5472.CAN-15-0159.

Mule J J, Shu S, Schwarz S L, Rosenberg S A. Adaptive immunotherapy of established pulmonary metastases with LAK cells and recombinant interleukin-2. Science 1984; 225(4669):1487-9.

Shultz L D, Lyons B L, Burzenski L M, Gott B, Chen X. Chaleff S, et al. Human lymphoid and myeloid cell development in NOD/LtSz-scid IL2R gamma null mice engrafted with mobilized human hemopoietic stem cells. J Immunol 2005; 174(10):6477-89

Xu X J, Song D G, Poussin M, Ye Q, Sharma P, Rodriguez-Garcia A, et al. Multiparameter comparative analysis reveals differential impacts of various cytokines on CART cell phenotype and function ex vivo and in vivo. Oncotarget 2016 doi 10.18632/oncotarget.10510.

Muranski P, Boni A, Wrzesinski C, Citrin D E, Rosenberg S A, Childs R, et al. Increased intensity lymphodepletion and adoptive immunotherapy—how far can we go? Nat Clin Tract Oncol 2006; 3(12):668-81 doi 10.1038/ncponc0666.

Gattinoni L. Finkelstein S E, Klebanoff C A, Antony P A, Palmer D C, Spiess P J, et al. Removal of homeostatic cytokine sinks by lymphodepletion enhances the efficacy of adoptively transferred tumor-specific CD8+ T cells. J Exp Med 2005; 202(7):907-12 doi 10.1084/jem.20050732.

Huang A Y, Gulden P H, Woods A S, Thomas M C, Tong C D, Wang W, et al. The immunodominant major histocompatibility complex class I-restricted antigen of a murine colon tumor derives from an endogenous retroviral gene product. Proc Natl Acad Sci USA 1996; 93(18):9730-5.

Beatty G L, Haas A R, Maus M V, Torigian D A, Soulen M C, Plesa G, et al. Mesothelin-specific chimeric antigen receptor mRNA-engineered T cells induce anti-tumor activity in solid malignancies. Cancer Immunol Res 2014; 2(2); 112-20 doi 10.1158/2326-6066.CIR-13-0170.

Casucci M, Bondanza A. Suicide gene therapy to increase the safety of chimeric antigen receptor-redirected T lymphocytes. J Cancer 2011; 2:378-82.

Rosenberg S A. Decade in review-cancer immunotherapy: Entering the mainstream of cancer treatment. Nature reviews Clinical oncology 2014 doi 10.1038/nrclinonc.2014.174.

1. A protein comprising an 5F9 anti-GCC scFV sequence selected from the group consisting of SEQ ID NO:8, SEQ ID NO:9, SEQ ID NO:10, SEQ ID NO:11, SEQ ID NO:12, SEQ ID NO:13, SEQ ID NO:14 and SEQ ID NO:15.

2. The protein of claim 1 further comprising a signal sequence, a hinge domain, a transmembrane domain, and a signaling domain.

3. The protein of claim 2 further comprising a signal sequence, a hinge domain, a transmembrane domain, and a signaling domain.

4. The protein of claim 3 wherein the signal sequence is selected from the group consisting of: a GM-CSF signal sequence, a CD8 alpha signal sequence, a CD8 beta signal sequence, a CD4 signal sequence, a TCR alpha signal sequence, a TCR beta signal sequence, a CD3 delta signal sequence, a CD3 epsilon signal sequence, a CD3 gamma signal sequence, a CD28 signal sequence, and a BiP signal sequence.

5. The protein of any of claims 2 to 4 wherein the hinge region is selected from the group consisting of: a CD8α hinge region, an IgG1-Fc hinge region, an IgG4-Fc hinge region, and a CD28 hinge region.

6. The protein of any of claims 2 to 5 wherein transmembrane region is selected from the group consisting of: a CD8α transmembrane region, an IgG4-Fc transmembrane region, an IgG4-Fc transmembrane region, and a CD28 transmembrane region.

7. The protein of any of claims 2 to 6 wherein the signaling domain is selected from the group consisting of: a CD28 signaling domain, a 4-1BB (CD137) signaling domain, a CD2 signaling domain, a CD27 signaling domain, a CD30 signaling domain, a CD40L signaling domain, a CD79A signaling domain, a CD79B signaling domain, a CD226 signaling domain, a DR3 signaling domain, a GITR signaling domain, a HVEM signaling domain, a ICOS signaling domain, a LIGHT signaling domain, a OX40 signaling domain, and a SLAM signaling domain.

8. The protein of any of claims 2 to 7 further comprising at least one immunoreceptor tyrosine activation motif (ITAM).

9. The protein of claim 8 comprising intracellular sequences that include ITAMs from CD3ζ, CD79-alpha, CD79-beta, or Fc receptor.

10. The protein of any of claims 1-9 further comprising an affinity tag.

11. The protein of claim 1 further comprising a CD8α hinge domain, a CD28 transmembrane domain, and a signaling domain comprising 4-1BB intracellular sequences and CO3ζ intracellular sequences.

12. The protein of claim 11 further comprising a GM-CSF signal sequence.

13. The protein of claim 12 having SEQ ID NO:2.

14. A nucleic acid molecule comprising a nucleic acid sequence that encodes a protein of any of claims 1-12.

15. A nucleic acid molecule comprising a nucleic acid sequence that encodes a protein of claims 12.

16. The nucleic acid molecule of claim 15 wherein nucleic acid sequence that encodes the protein is operably linked to regulatory elements for expression in human T cells.

17. A recombinant cell comprising the nucleic acid molecule of claim 16.

18. A recombinant T cell comprising the nucleic acid molecule of claim 16.

19. The nucleic acid molecule of claim 13 comprising SEQ ID NO:1.

20. The nucleic acid molecule of claim 19 wherein SEQ ID NO:1 is operably linked to regulatory elements for expression in human T cells.

21. A recombinant cell comprising the nucleic acid molecule of claim 20.

22. A recombinant T cell comprising the nucleic acid molecule of claim 20.

23. A recombinant cell comprising the nucleic acid molecule of claim 15.

24. A recombinant T cell comprising the nucleic acid molecule of claim 15.

25. A recombinant cell comprising the protein of any of claims 1-15.

26. A recombinant T cell comprising the protein of any of claims 1-15.

27. A recombinant cell comprising the protein of claim 11.

28. A recombinant T cell comprising the protein of claim 11.

29. A recombinant cell comprising the protein of claim 13.

30. A recombinant T cell comprising the protein of claim 13.

31. A method of treating a patient who has cancer that has cancer cells that express GUCY2C, the method comprises the step of administering to said patient the plurality of recombinant cells of any of claims 17, 18 and 21 to 30.

32. The method of claim 31 wherein the plurality of recombinant cells is a plurality of recombinant T cells.

33. A method of treating a patient who has cancer that has cancer cells that express GUCY2C, the method comprises the steps of: isolating T cells from the patient; transforming the T cells with a nucleic acid molecule of claim 20 to produce a population of transformed T cells that express SEQ ID NO:1 and comprise SEQ ID NO:2 as a membrane bound protein, expanding the population of transformed T cells to produce a plurality of transformed T cells, and administering to said patient the plurality of recombinant T cells.

34. The method of any of claims 31 or 33 wherein prior to isolating cells from the patient, a sample of cancer cells is isolated from the patient and GUCY2C is detected on said cancer cells.

35. A method of preventing cancer that has cancer cells that express GUCY2C in a patient identified as being of increased risk, the method comprises the step of administering to said patient the plurality of recombinant cells of any of claims 17, 18 and 21 to 30.

36. The method of claim 35 wherein the plurality of recombinant cells is a plurality of recombinant T cells.

37. A method of preventing cancer that has cancer cells that express GUCY2C in a patient identified as being of increased risk, the method comprises the steps of: isolating T cells from the patient; transforming the T cells with a nucleic acid molecule of claim 20 to produce a population of transformed T cells that express SEQ ID NO:1 and comprise SEQ ID NO:2 as a membrane bound protein, expanding the population of transformed T cells to produce a plurality of transformed T cells, and administering to said patient the plurality of transformed T cells.

38. A method of making the plurality of recombinant cells of claim 21 comprising the steps of: isolating cells from an individual; transforming the cells with a nucleic acid molecule that encodes SEQ ID NO:2 operable linked to regulatory elements functional in cells to produce a population of transformed cells that comprise SEQ ID NO:2 as a membrane bound protein, and expanding the population of transformed cells to produce a plurality of recombinant cells.

39. A method of making the plurality of recombinant T cells of claim 22 comprising the steps of: isolating T cells from an individual; transforming the T cells with a nucleic acid molecule that encodes SEQ ID NO:2 operable linked to regulatory elements functional in T cells to produce a population of transformed T cells that comprise SEQ ID NO:2 as a membrane bound protein, and expanding the population of transformed T cells to produce a plurality of recombinant T cells.


SEQUENCE LISTING (15 sequences)

SEQ ID NO: 1 - DNA, 1671 bp, Artificial Sequence
  Synthetic CAR-T containing human derived sequences
  CDS (1)..(1671): 5F9.28BBz CAR construct for human CAR-T cell expression
  sig_peptide (1)..(66): hGMCSFRa Signal Peptide
  misc_feature (73)..(822): 5F9 anti-GUYC2C scFv
  misc_feature (829)..(1008): CD8 hinge
  misc_feature (1009)..(1215): CD28 TM
  misc_feature (1216)..(1332): 4-1BB intracellular - signalling domain
  misc_feature (1333)..(1671): CD3zeta intracellular - signalling domain ITAM
atgctgctgctcgtgacatctctgctgctgtgcgagctgccccaccccgcctttctgctgatccccgatatcgagatcgtgatgacccagagccccgccaccctgagtgtgtctccaggcgaaagagccaccctgtcctgcagagccagccagagcgtgtccagaaacctggcctggtatcagcagaagcccggccaggctccccggctgctgatctatggcgccagcacaagagccacaggcatccccgccagattttccggctctggcagcggcaccgagttcaccctgacaatcggcagcctgcagtccgaggacttcgccgtgtactactgccagcagtacaagacctggccccggacctttggccagggcaccaacgtggaaatcaaggcctctggcggcggaggatctgggggaggcggaagcggaggcggaggaagtggcggagggggatctgaactgcaggtgcagctgcagcagtggggagccggactgctgaagcctagcgagacactgagcctgacctgcgccgtgttcggcggcagcttcagcggctactactggtcctggatcagacagccccctggcaagggcctggaatggatcggcgagatcaaccaccggggcaacaccaacgacaaccccagcctgaagtccagagtgaccatcagcgtggacaccagcaagaaccagttcgccctgaagctgagcagcgtgacagccgccgataccgccgtgtattattgcgccagagagcggggctacacctacggcaacttcgaccactggggccagggaaccctcgtgaccgtgtctagcgagctcctgagcaacagcatcatgtacttcagccacttcgtgcccgtgtttctgcccgccaagcctaccacaacccctgcccctagacctcctaccccagcccctacaatcgcctcccagcctctgtctctgaggcccgaggcttgtagacctgctgctggcggagccgtgcacaccagaggactgttttgggtgctggtggtcgtgggcggagtgctggcctgttacagcctgctcgtgactgtggccttcatcatcttttgggtgcgcagcaagcggagcagactgctgcacagcgactacatgaacatgacccccagacggccaggccccaccagaaagcactaccagccttacgcccctccccgggatttcgccgcctacagaagcggcagaaagaagctgctgtacatcttcaagcagcccttcatgcggcccgtgcagaccacccaggaagaggacggctgctcctgcagattccccgaggaagaagaaggcggctgcgagctgagagtgaagttcagcagatccgccgacgcccctgcctatcagcagggccagaaccagctgtacaacgagctgaacctgggcagacgggaagagtacgacgtgctggacaagcggagaggcagggaccctgagatgggcggaaagccccagagaagaaagaacccccaggaaggcctgtataacgaactgcagaaagacaagatggccgaggcctacagcgagatcggaatgaagggcgagcggagaagaggcaagggacacgacggcctgtaccagggactgagcaccgccaccaaggacacctacgacgccctgcacatgcaggccctgccccccaga

SEQ ID NO: 2 - protein, 557 aa, Artificial Sequence
  Synthetic Construct
MLLLVTSLLLCELPHPAFLLIPDIEIVMTQSPATLSVSPGERATLSCRASQSVSRNLAWYQQKPGQAPRLLIYGASTRATGIPARFSGSGSGTEFTLTIGSLQSEDFAVYYCQQYKTWPRTFGQGTNVEIKASGGGGSGGGGSGGGGSGGGGSELQVQLQQWGAGLLKPSETLSLTCAVFGGSFSGYYWSWIRQPPGKGLEWIGEINHRGNTNDNPSLKSRVTISVDTSKNQFALKLSSVTAADTAVYYCARERGYTYGNFDHWGQGTLVTVSSELLSNSIMYFSHFVPVFLPAKPTTTPAPRPPTPAPTIASQPLSLRPEACRPAAGGAVHTRGLFWVLVVVGGVLACYSLLVTVAFIIFWVRSKRSRLLHSDYMNMTPRRPGPTRKHYQPYAPPRDFAAYRSGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 3 - protein, 5 aa, Artificial Sequence
  Synthetic linker sequence for scFV
GGGGS

SEQ ID NO: 4 - protein, 10 aa, Artificial Sequence
  Synthetic linker sequence for scFV
GGGGSGGGGS

SEQ ID NO: 5 - protein, 15 aa, Artificial Sequence
  Synthetic linker sequence for scFV
GGGGSGGGGSGGGGS

SEQ ID NO: 6 - protein, 20 aa, Artificial Sequence
  Synthetic linker sequence for scFV
GGGGSGGGGSGGGGSGGGGS

SEQ ID NO: 7 - protein, 25 aa, Artificial Sequence
  Synthetic linker sequence for scFV
GGGGSGGGGSGGGGSGGGGSGGGGS

SEQ ID NO: 8 - protein, 240 aa, Artificial Sequence
  Synthetic construct scFv VL-L2-VH
  MISC_FEATURE (1)..(109): 5F9 Variable Light Chain fragment
  MISC_FEATURE (110)..(119): Linker G4S-2
  MISC_FEATURE (120)..(240): 5F9 Variable Heavy Chain fragment
EIVMTQSPATLSVSPGERATLSCRASQSVSRNLAWYQQKPGQAPRLLIYGASTRATGIPARFSGSGSGTEFTLTIGSLQSEDFAVYYCQQYKTWPRTFGQGTNVEIKASGGGGSGGGGSELQVQLQQWGAGLLKPSETLSLTCAVFGGSFSGYYWSWIRQPPGKGLEWIGEINHRGNTNDNPSLKSRVTISVDTSKNQFALKLSSVTAADTAVYYCARERGYTYGNFDHWGQGTLVTVSS

SEQ ID NO: 9 - protein, 245 aa, Artificial Sequence
  Synthetic construct scFv VL-L3-VH
  MISC_FEATURE (1)..(109): 5F9 Variable Light Chain fragment
  MISC_FEATURE (110)..(124): Linker G4S-3
  MISC_FEATURE (125)..(245): 5F9 Variable Heavy Chain fragment
EIVMTQSPATLSVSPGERATLSCRASQSVSRNLAWYQQKPGQAPRLLIYGASTRATGIPARFSGSGSGTEFTLTIGSLQSEDFAVYYCQQYKTWPRTFGQGTNVEIKASGGGGSGGGGSGGGGSELQVQLQQWGAGLLKPSETLSLTCAVFGGSFSGYYWSWIRQPPGKGLEWIGEINHRGNTNDNPSLKSRVTISVDTSKNQFALKLSSVTAADTAVYYCARERGYTYGNFDHWGQGTLVTVSS

SEQ ID NO: 10 - protein, 250 aa, Artificial Sequence
  Synthetic construct scFv VL-L4-VH
  MISC_FEATURE (1)..(109): 5F9 Variable Light Chain fragment
  MISC_FEATURE (110)..(129): Linker G4S-4
  MISC_FEATURE (130)..(250): 5F9 Variable Heavy Chain fragment
EIVMTQSPATLSVSPGERATLSCRASQSVSRNLAWYQQKPGQAPRLLIYGASTRATGIPARFSGSGSGTEFTLTIGSLQSEDFAVYYCQQYKTWPRTFGQGTNVEIKASGGGGSGGGGSGGGGSGGGGSELQVQLQQWGAGLLKPSETLSLTCAVFGGSFSGYYWSWIRQPPGKGLEWIGEINHRGNTNDNPSLKSRVTISVDTSKNQFALKLSSVTAADTAVYYCARERGYTYGNFDHWGQGTLVTVSS

SEQ ID NO: 11 - protein, 255 aa, Artificial Sequence
  Synthetic construct scFv VL-L5-VH
  MISC_FEATURE (1)..(109): 5F9 Variable Light Chain fragment
  MISC_FEATURE (110)..(134): Linker G4S-5
  MISC_FEATURE (135)..(255): 5F9 Variable Heavy Chain fragment
EIVMTQSPATLSVSPGERATLSCRASQSVSRNLAWYQQKPGQAPRLLIYGASTRATGIPARFSGSGSGTEFTLTIGSLQSEDFAVYYCQQYKTWPRTFGQGTNVEIKASGGGGSGGGGSGGGGSGGGGSGGGGSELQVQLQQWGAGLLKPSETLSLTCAVFGGSFSGYYWSWIRQPPGKGLEWIGEINHRGNTNDNPSLKSRVTISVDTSKNQFALKLSSVTAADTAVYYCARERGYTYGNFDHWGQGTLVTVSS

SEQ ID NO: 12 - protein, 240 aa, Artificial Sequence
  Synthetic construct scFv VH-L2-VL
  MISC_FEATURE (1)..(121): 5F9 Variable Heavy Chain fragment
  MISC_FEATURE (122)..(131): Linker G4S-2
  MISC_FEATURE (132)..(240): 5F9 Variable Light Chain fragment
ELQVQLQQWGAGLLKPSETLSLTCAVFGGSFSGYYWSWIRQPPGKGLEWIGEINHRGNTNDNPSLKSRVTISVDTSKNQFALKLSSVTAADTAVYYCARERGYTYGNFDHWGQGTLVTVSSGGGGSGGGGSEIVMTQSPATLSVSPGERATLSCRASQSVSRNLAWYQQKPGQAPRLLIYGASTRATGIPARFSGSGSGTEFTLTIGSLQSEDFAVYYCQQYKTWPRTFGQGTNVEIKAS

SEQ ID NO: 13 - protein, 245 aa, Artificial Sequence
  Synthetic construct scFv VH-L3-VL
  MISC_FEATURE (1)..(121): 5F9 Variable Heavy Chain fragment
  MISC_FEATURE (122)..(136): Linker G4S-3
  MISC_FEATURE (137)..(245): 5F9 Variable Light Chain fragment
ELQVQLQQWGAGLLKPSETLSLTCAVFGGSFSGYYWSWIRQPPGKGLEWIGEINHRGNTNDNPSLKSRVTISVDTSKNQFALKLSSVTAADTAVYYCARERGYTYGNFDHWGQGTLVTVSSGGGGSGGGGSGGGGSEIVMTQSPATLSVSPGERATLSCRASQSVSRNLAWYQQKPGQAPRLLIYGASTRATGIPARFSGSGSGTEFTLTIGSLQSEDFAVYYCQQYKTWPRTFGQGTNVEIKAS

SEQ ID NO: 14 - protein, 250 aa, Artificial Sequence
  Synthetic construct scFv VH-L4-VL
  MISC_FEATURE (1)..(121): 5F9 Variable Heavy Chain fragment
  MISC_FEATURE (122)..(141): Linker G4S-4
  MISC_FEATURE (142)..(250): 5F9 Variable Light Chain fragment
ELQVQLQQWGAGLLKPSETLSLTCAVFGGSFSGYYWSWIRQPPGKGLEWIGEINHRGNTNDNPSLKSRVTISVDTSKNQFALKLSSVTAADTAVYYCARERGYTYGNFDHWGQGTLVTVSSGGGGSGGGGSGGGGSGGGGSEIVMTQSPATLSVSPGERATLSCRASQSVSRNLAWYQQKPGQAPRLLIYGASTRATGIPARFSGSGSGTEFTLTIGSLQSEDFAVYYCQQYKTWPRTFGQGTNVEIKAS

SEQ ID NO: 15 - protein, 255 aa, Artificial Sequence
  Synthetic construct scFv VH-L5-VL
  MISC_FEATURE (1)..(121): 5F9 Variable Heavy Chain fragment
  MISC_FEATURE (122)..(146): Linker G4S-5
  MISC_FEATURE (147)..(255): 5F9 Variable Light Chain fragment
ELQVQLQQWGAGLLKPSETLSLTCAVFGGSFSGYYWSWIRQPPGKGLEWIGEINHRGNTNDNPSLKSRVTISVDTSKNQFALKLSSVTAADTAVYYCARERGYTYGNFDHWGQGTLVTVSSGGGGSGGGGSGGGGSGGGGSGGGGSEIVMTQSPATLSVSPGERATLSCRASQSVSRNLAWYQQKPGQAPRLLIYGASTRATGIPARFSGSGSGTEFTLTIGSLQSEDFAVYYCQQYKTWPRTFGQGTNVEIKAS